(19) United States
(12) Patent Application Publication
(10) Pub. No.: US 2023/0363766 A1
(43) Pub. Date: Nov. 16, 2023

(54) COMPRESSION DEVICE SET AND ADHERING METHOD

(71) Applicant: TERUMO KABUSHIKI KAISHA, Tokyo (JP)

(72) Inventor: Masakatsu KAWAURA, Kanagawa (JP)

(73) Assignee: TERUMO KABUSHIKI KAISHA, Tokyo (JP)

(21) Appl. No.: 18/360,921

(22) Filed: Jul. 28, 2023

Related U.S. Application Data
(63) Continuation of application No. PCT/JP2022/010144, filed on Mar. 8, 2022.

(30) Foreign Application Priority Data
Mar. 15, 2021 (JP) 2021-041840

(51) Int. Cl. A61B 17/132; A61F 13/02

(52) U.S. Cl. CPC A61B 17/1325; A61F 13/02

(57) ABSTRACT

A compression device set according to the present disclosure includes a compression device configured to be adhered to a biological surface and to compress the biological surface in a state of being adhered to the biological surface; and a pre-compression member attached to the compression device and configured to compress, before the compression device is adhered, the biological surface to which the compression device is to be adhered. The compression device includes an adhesion surface configured to be adhered to the biological surface, and a compression portion configured to compress the biological surface in a compression region that is sandwiched by the adhesion surface or surrounded by the adhesion surface. The pre-compression member includes a protruding portion protruding from the adhesion surface of the compression device and configured to compress the biological surface before the adhesion surface is adhered to the biological surface in the compression region.

### CROSS-REFERENCES TO RELATED APPLICATIONS

This application is a continuation of International Application No. PCT/JP2022/010144 filed on Mar. 8, 2022, which claims priority to Japanese Application No. 2021-041840 filed on Mar. 15, 2021, the entire content of both of which is incorporated herein by reference.

### TECHNOLOGICAL FIELD

The present disclosure relates to a compression device set and an adhering method.

### BACKGROUND DISCUSSION

In recent years, in medical institutions, various forms of examinations or treatments have been performed using catheters. The catheters can be, for example, percutaneously inserted into a blood vessel from a puncture site formed at a wrist, an inguinal region, or the like, and is carried through the blood vessel to a site to be diagnosed or treated. After a diagnosis or treatment performed by a health care worker is completed, an elongated insertion member such as a puncture needle, a catheter, and a sheath used for introducing the catheter into a living body is removed from a puncture site, and the puncture site is stopped from bleeding.

International Patent Application Publication No. WO2003/082127A (which corresponds to Japanese Patent Application Publication No. 2005-521464A) discloses a dressing as a compression device that compresses a wound of a patient after removing a sheath. The dressing in International Patent Application Publication No. WO2003/082127A includes an inflatable bladder having a deflated state in which a membrane is adjacent to an end wall and an inflated state in which the membrane is spaced from the end wall. In addition, the dressing in International Patent Application Publication No. WO2003/082127A includes a holding portion that holds the bladder against a skin of the patient at a position at which the wound is substantially covered. International Patent Application Publication No. WO2003/082127A discloses the holding portion including a flexible web that is connected to the end wall of the bladder and that protrudes outward from the end wall of the bladder, and one surface of the flexible web is provided with an adhesive layer for being adhered to the skin of the patient.

In the dressing serving as the compression device described in International Patent Application Publication No. WO2003/082127A, the wound of the patient can be compressed by the bladder by adhering the adhesive layer provided on the one surface of the flexible web to the skin serving as a biological surface of the patient and keeping the bladder in an inflated state.

However, regarding the dressing described in International Patent Application Publication No. WO2003/082127A, when the wound of the patient is compressed by the bladder, the adhesive layer provided on the one surface of the flexible web may be peeled off from the skin by depression of the skin due to the compression and a reaction force received from the skin. When the adhesive layer is peeled off from the skin, a desired compression force from the bladder may not be obtained.

### SUMMARY

An adhering method and a compression device set are disclosed in which a compression device is less likely to be peeled off from a biological surface.

A compression device set according to a first aspect of the present disclosure comprises: a compression device configured to be adhered to a biological surface and to compress the biological surface in a state of being adhered to the biological surface; and a pre-compression member attached to the compression device and configured to compress, before the compression device is adhered, the biological surface to which the compression device is to be adhered. The compression device comprises an adhesion surface configured to be adhered to the biological surface, and a compression portion configured to compress the biological surface in a compression region that is a position sandwiched by the adhesion surface or a position surrounded by the adhesion surface in a plan view viewed along a direction orthogonal to the adhesion surface. The pre-compression member includes a protruding portion protruding to a direction of the biological surface from the adhesion surface of the compression device and configured to compress the biological surface before the adhesion surface is adhered to the biological surface in the compression region.

According to one embodiment of the present disclosure, the pre-compression member is attached to the compression device in a manner of being relatively movable in a direction orthogonal to the adhesion surface.

According to one embodiment of the present disclosure, the compression device and the pre-compression member include a movement restriction mechanism configured to limit movement of the pre-compression member relative to the compression device in the direction orthogonal to the adhesion surface and configured to fix a minimum protrusion amount of the protruding portion.

According to one embodiment of the present disclosure, the movement restriction mechanism is configured to adjust the minimum protrusion amount of the protruding portion in a plurality of stages.

According to one embodiment of the present disclosure, the pre-compression member is attachable to and detachable from the compression device.

According to one embodiment of the present disclosure, the pre-compression member includes, as the protruding portion, a first protruding portion and a second protruding portion that are provided on both sides sandwiching the compression portion of the compression device in the plan view viewed along the direction orthogonal to the adhesion surface.

According to one embodiment of the present disclosure, the pre-compression member further includes, as the protruding portion, a third protruding portion provided on the compression portion of the compression device in a direction orthogonal to a direction in which the first protruding portion and the second protruding portion face each other in the plan view viewed along the direction orthogonal to the adhesion surface.

According to one embodiment of the present disclosure, the compression device further includes an adhesion body having the adhesion surface, and a compression member fixed to a surface opposite to the adhesion surface of the adhesion body, the compression member includes an inflatable portion constituting the compression portion and being inflatable, and a support portion configured to support the inflatable portion, the support portion defines a through hole penetrating in the direction orthogonal to the adhesion surface, and the protruding portion of the pre-compression member protrudes from the adhesion surface through the through hole.

A compression device set according to a second aspect of the present disclosure includes: a compression device configured to be adhered to a biological surface; a pre-compression member to compress, before the compression device is adhered, the biological surface to which the compression device is to be adhered; the compression device including an adhesion surface configured to be adhered to the biological surface and a compression portion configured to compress the biological surface in a compression region; and wherein the pre-compression member includes a protruding portion configured to protrude in a direction of the biological surface from the adhesion surface of the compression device and configured to compress the biological surface before the adhesion surface is adhered to the biological surface in the compression region.

An adhering method according to a third aspect of the present disclosure is an adhering method for adhering a compression device configured to compress the biological surface in a state of being adhered to the biological surface to the biological surface in a state of pre-compressing the biological surface. The compression device includes an adhesion surface configured to be adhered to the biological surface, and a compression portion configured to compress the biological surface in a compression region that is a position sandwiched by the adhesion surface or a position surrounded by the adhesion surface in a plan view viewed along a direction orthogonal to the adhesion surface. The adhering method includes adhering, in the compression region, the adhesion surface of the compression device to the biological surface in a state of compressing the biological surface by a protruding portion of a pre-compression member attached to the compression device, the protruding portion protruding from the adhesion surface.

According to the present disclosure, it is possible to provide the adhering method and the compression device set in which the compression device is less likely to be peeled off from the biological surface.

### BRIEF DESCRIPTION OF THE DRAWINGS

FIG. 1 is a side view showing a compression device set according to a first embodiment of the present disclosure.

FIG. 2 is an exploded side view of the compression device set shown in FIG. 1.

FIG. 3A is a view showing a state before a biological surface is pre-compressed by a pre-compression member of the compression device set shown in FIG. 1.

FIG. 3B is a view showing a state in which the biological surface is pre-compressed by the pre-compression member of the compression device set shown in FIG. 1.

FIG. 3C is a view showing a state in which an adhesion surface of a compression device of the compression device set shown in FIG. 1 is adhered to the biological surface in the pre-compression state shown in FIG. 3B.

FIG. 3D is a view showing a state in which the pre-compression on the biological surface performed by the pre-compression member of the compression device set shown in FIG. 1 is released from the state shown in FIG. 3C.

FIG. 3E is a view showing a state in which the biological surface is compressed by a compression portion of the compression device of the compression device set shown in FIG. 1 after the state shown in FIG. 3D.

FIG. 4 is a perspective view of a compression device set according to a second embodiment of the present disclosure as viewed from an upper surface side.

FIG. 5A is a top view of the compression device set shown in FIG. 4, and FIG. 5B is a bottom view of the compression device set shown in FIG. 4.

FIG. 6 is a side view of the compression device set shown in FIG. 4.

FIG. 7 is a cross-sectional view of the compression device set shown in FIG. 4 at a position taken along a line I-I in FIGS. 5A and 5B.

FIG. 8 is a cross-sectional view of the compression device set at the same position as in FIG. 7 in a state in which an inflatable portion of the compression device shown in FIG. 4 is inflated.

FIG. 9A is a view showing a state in which the pre-compression member of the compression device set shown in FIG. 4 is removed from the compression device, and FIG. 9B is a view showing a state in which the pre-compression member shown in FIG. 9A is fixed to the compression device at a position different from that in FIG. 4.

FIG. 10 is a perspective view of a compression device set according to a third embodiment of the present disclosure as viewed from an upper surface side.

FIG. 11 is a perspective view of the compression device set shown in FIG. 10 as viewed from a lower surface side.

FIG. 12A is a top view of the compression device set shown in FIG. 10, and FIG. 12B is a bottom view of the compression device set shown in FIG. 10.

FIG. 13 is a side view of the compression device set shown in FIG. 10, and shows a case in which an inflatable portion is in a deflated form.

FIG. 14A is a side view of the compression device set shown in FIG. 10, and shows a case in which the inflatable portion is in an inflated form, and FIG. 14B is a side view of the compression device set in a state in which a position of a pre-compression member is different from that in FIG. 14A.

FIG. 15 is a perspective view of the compression device set shown in FIG. 10 as viewed from the upper surface side, and shows a state in which the position of the pre-compression member is different from that in FIG. 10.

FIG. 16 is an exploded perspective view of the pre-compression member shown in FIG. 10.

FIG. 17 is a side view of the compression device set showing a state in which the pre-compression member is removed from the compression device in the compression device set shown in FIG. 10.

FIG. 18A is a diagram showing a state in which a medical insertion member is inserted into a femoral vein from a biological surface through a connective tissue.

FIG. 18B is a diagram showing a state after the medical insertion member is removed from the state shown in FIG. 18A.

FIG. 19 is a diagram showing a state in which perforations shown in FIG. 18B are narrowed or obstructed by the compression device shown in FIG. 4.

FIG. 20 is a front view of the state shown in FIG. 19 as viewed from the biological surface side.

### DETAILED DESCRIPTION

Hereinafter, embodiments of a compression device set and an adhering method according to the present disclosure will be exemplified and described with reference to the drawings. In the drawings, common configurations are denoted by the same reference signs.

### First Embodiment

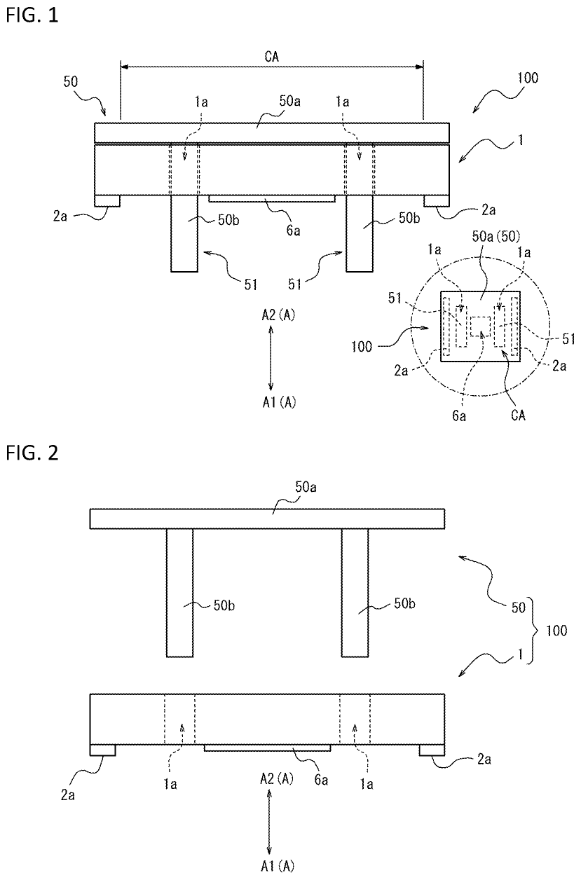

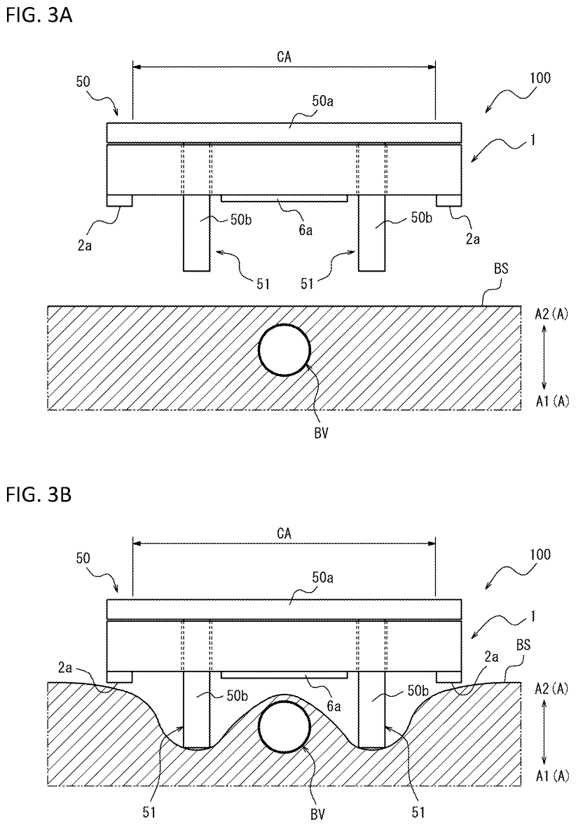

FIG. 1 is a side view showing a compression device set 100 according to an embodiment of the present disclosure. As shown in FIG. 1, the compression device set 100 includes a compression device 1 and a pre-compression member 50. FIG. 1 shows, in addition to the side view of the compression device set 100, a plan view of the compression device set 100 viewed along a direction orthogonal to adhesion surfaces 2a of the compression device 1 within a frame of a dashed line. FIG. 2 is an exploded side view of the compression device set 100 shown in FIG. 1. Specifically, FIG. 2 shows a state in which the compression device 1 and the pre-compression member 50 of the compression device set 100 shown in FIG. 1 are spaced apart and separated from each other.

The compression device 1 can be adhered to a biological surface. The compression device 1 can compress the biological surface in a state of being adhered to the biological surface.

Specifically, the compression device 1 has adhesion surfaces 2a that can be adhered to the biological surface. The compression device 1 can be attached to the biological surface by the adhesion surfaces 2a being adhered to the biological surface. The compression device 1 also includes a compression portion 6a that compresses the biological surface in a compression region CA in a state in which the adhesion surfaces 2a are adhered to the biological surface. The “compression region CA” means a position sandwiched by the adhesion surfaces 2a or a position surrounded by the adhesion surfaces 2a in a plan view viewed along a direction orthogonal to the adhesion surfaces 2a. The phrase “a position sandwiched by the adhesion surfaces 2a in a plan view viewed along a direction orthogonal to the adhesion surfaces 2a” is not limited to a position sandwiched by a plurality of adhesion surfaces 2a spaced apart from each other in the same plan view, and may be a position sandwiched by a part of one adhesion surface 2a continuous in the same plan view, such as a substantially U-shape or a substantially C-shape in the same plan view. In addition, the phrase “a position surrounded by the adhesion surface 2a in a plan view viewed along a direction orthogonal to the adhesion surface 2a” is not limited to a position at which an entire periphery is surrounded by the adhesion surface 2a in the same plan view, and may be, for example, a position that is surrounded by a convex hull defined by three or more adhesion surfaces 2a spaced apart from each other in the same plan view, or may be a position surrounded by one adhesion surface 2a having a gap in part, such as a substantially U-shape or a substantially C-shape in the same plan view.

Among the directions orthogonal to the adhesion surface 2a, a direction from a surface opposite to the surface on which the adhesion surface 2a is provided toward the surface on which the adhesion surface 2a is provided is an adhesion direction in which the compression device 1 is adhered to the biological surface, and hereinafter, may be simply referred to as a “downward direction A1” or a “lower side” for convenience of description. Among the directions orthogonal to the adhesion surface 2a, a direction opposite to the downward direction A1 is a separation direction in which the compression device 1 is spaced apart from the biological surface, and hereinafter, may be simply referred to as an “upward direction A2” or an “upper side” for convenience of description.

The compression portion 6a according to the present embodiment can compress the biological surface at the position sandwiched by the adhesion surfaces 2a in a plan view (see the inside of the frame of the dashed line in FIG. 1) of the compression device set 100 viewed along the direction orthogonal to the adhesion surface 2a.

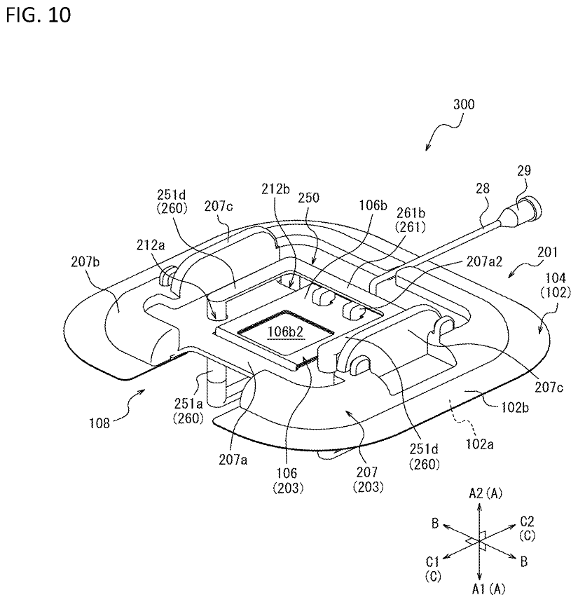

As shown in FIG. 1, the pre-compression member 50 is attached to the compression device 1. The pre-compression member 50 according to the present embodiment may be removed from the compression device 1 without changing a form of the pre-compression member 50, and is not limited to this configuration. As described later, the pre-compression member 50 may be attachable to and detachable from the compression device 1 by changing the form of the pre-compression member 50 (see FIGS. 9, 16, and 17). The pre-compression member 50 may not be detachable from the compression device 1.

The pre-compression member 50 can compress the biological surface to which the compression device 1 is to be adhered before the compression device 1 is adhered. More specifically, the pre-compression member 50 can include, in the compression region CA, protruding portions 51 that protrude downward from the adhesion surfaces 2a of the compression device 1 and that can compress the biological surface before the adhesion surface 2a is adhered to the biological surface. The protruding portions 51 according to the present embodiment may compress the biological surface at positions between the adhesion surfaces 2a and the compression portion 6a in the compression region CA in the plan view (see the inside of the frame of the dashed line in FIG. 1) of the compression device set 100 viewed along the direction orthogonal to the adhesion surface 2a.

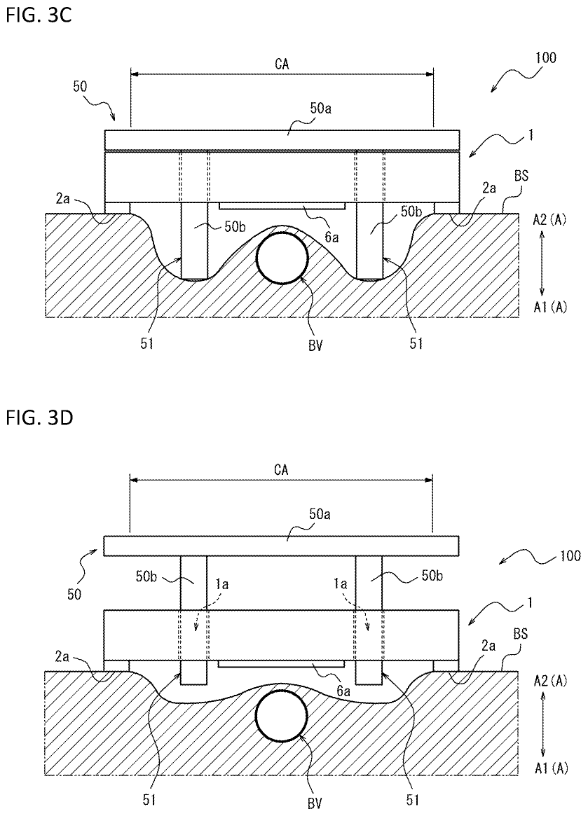

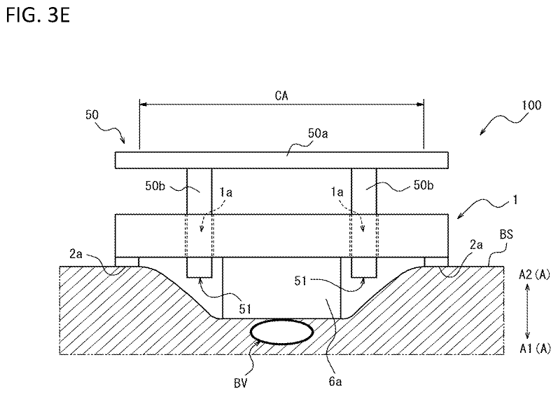

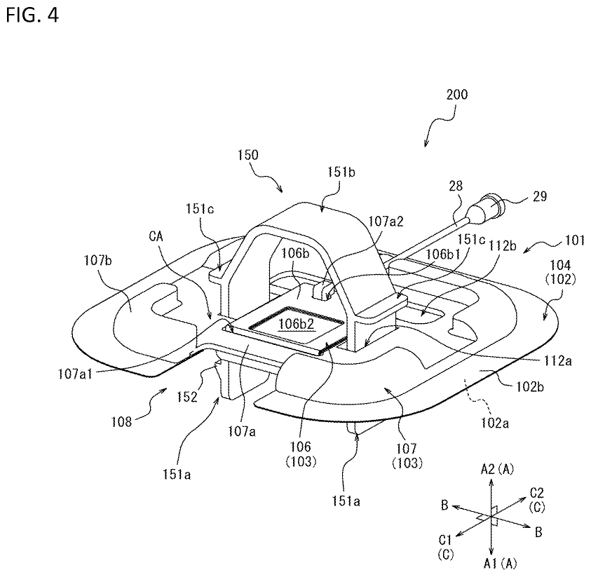

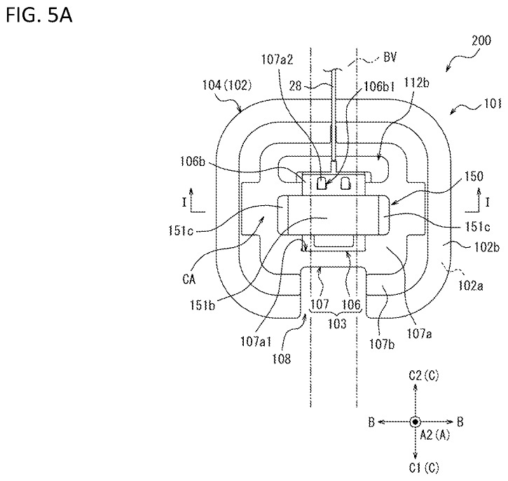

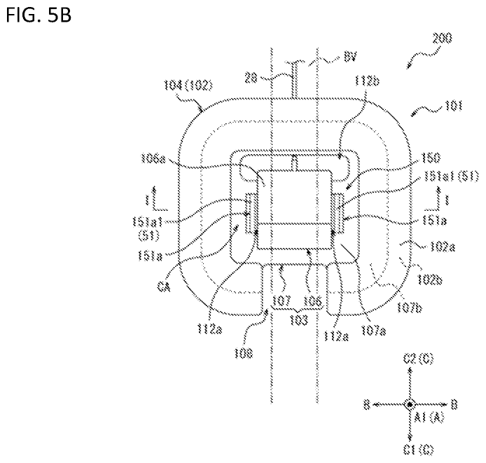

FIGS. 3A to 3E are schematic views showing an example of an adhering method for adhering the compression device 1 to a biological surface BS by the compression device set 100. Specifically, FIG. 3A is a view showing a state before the biological surface BS is pre-compressed by the pre-compression member 50 of the compression device set 100. FIG. 3B is a view showing a state in which the biological surface BS is pre-compressed by the pre-compression member 50 of the compression device set 100 shown in FIG. 1. FIG. 3C is a view showing a state in which the adhesion surface 2a of the compression device 1 of the compression device set 100 is adhered to the biological surface BS in the pre-compression state shown in FIG. 3B. FIG. 3D is a view showing a state in which the pre-compression on the biological surface BS performed by the pre-compression member 50 of the compression device set 100 is released from the state shown in FIG. 3C. FIG. 3E is a view showing a state in which the biological surface BS is compressed by the compression portion 6a in the compression device 1 of the compression device set 100 and a blood vessel BV is narrowed or obstructed after the state shown in FIG. 3D.

As shown in FIGS. 3A and 3B, according to the compression device set 100, when the adhesion surface 2a of the compression device 1 is adhered to the biological surface BS, the protruding portions 51 of the pre-compression member 50 protruding from the adhesion surface 2a compress the biological surface BS before the adhesion surface 2a is adhered to the biological surface BS. Hereinafter, compressing the biological surface BS by the protruding portions 51 of the pre-compression member 50 before the adhesion surface 2a of the compression device 1 is adhered to the biological surface BS is referred to as “pre-compression”. As shown in FIG. 3C, the adhesion surface 2a of the compression device 1 is adhered to the biological surface BS in a state in which the protruding portions 51 of the pre-compression member 50 pre-compress the biological surface BS in the compression region CA. In other words, in the compression region CA, the adhesion surface 2a of the compression device 1 is adhered to the biological surface BS in a state in which the biological surface BS is compressed by the protruding portions 51 protruding from the adhesion surface 2a and provided in the pre-compression member 50 attached to the compression device 1. As shown in FIG. 3D, the protruding portions 51 of the pre-compression member 50 pre-compress the biological surface BS in the compression region CA, so that slack (i.e., excess skin of the biological surface) can be formed in the region of the biological surface BS sandwiched or surrounded by the adhesion surface 2a adhered to the biological surface BS thereafter.

When there is no slack in the region of the biological surface BS sandwiched or surrounded by the adhesion surface 2a adhered to the biological surface BS, a skin of the biological surface BS is pulled in a manner of stretching along the biological surface BS when the biological surface BS is compressed by the compression portion 6a. Therefore, a shearing force along the biological surface BS is likely to act between the adhesion surface 2a of the compression device 1 and the biological surface BS. Due to the shearing force, the adhesion surface 2a of the compression device 1 is likely to be peeled off from the biological surface BS. However, when the slack is formed in the region of the biological surface BS sandwiched or surrounded by the adhesion surface 2a adhered to the biological surface BS, as shown in FIG. 3E, the biological surface BS is pressed in a concave shape such that the slack is stretched when the biological surface BS is compressed by the compression portion 6a. Therefore, the skin of the biological surface BS is less likely to be pulled in a manner of extending along the biological surface BS. That is, the shearing force along the biological surface BS is less likely to act between the adhesion surface 2a of the compression device 1 and the biological surface BS. As a result, the adhesion surface 2a of the compression device 1 is less likely to be detached from the biological surface BS. That is, according to the compression device set 100, it is possible to implement the compression device 1 which is less likely to be peeled off from the biological surface BS.

The pre-compression member 50 according to the present embodiment can include a plate-shaped main body portion 50a that covers an outer surface opposite to the adhesion surface 2a in the compression device 1, and insertion portions 50b that extend from the main body portion 50a to an adhesion surface 2a side of the compression device 1 through through holes 1a defined by the compression device 1. In the present embodiment, the protruding portions 51 protruding from the adhesion surface 2a of the compression device 1 include tip portions of the insertion portions 50b. However, specific configurations of the compression device 1 and the pre-compression member 50 are not limited to configurations according to the present embodiment.

As shown in FIGS. 3B to 3E, the projecting portions 51 of the pre-compression member 50 according to the present embodiment compress the biological surface at the position between the adhesion surface 2a and the compression portion 6a in the compression region CA in the plan view (see the inside of the frame of the dashed line in FIG. 1) of the compression device set 100 viewed along the direction orthogonal to the adhesion surface 2a. The protruding portions 51 of the pre-compression member 50 compresses the biological surface at the position between the adhesion surface 2a and the compression portion 6a, so that the pre-compression member 50 is less likely to interfere with adhering the adhesion surface 2a to the biological surface and compressing the biological surface by the compression portion 6a. However, the position at which the protruding portion 51 of the pre-compression member 50 compresses the biological surface may be another position within the compression region CA.

### Second Embodiment

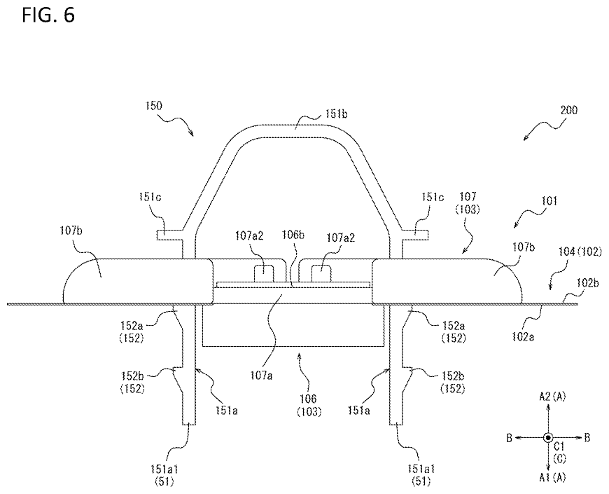

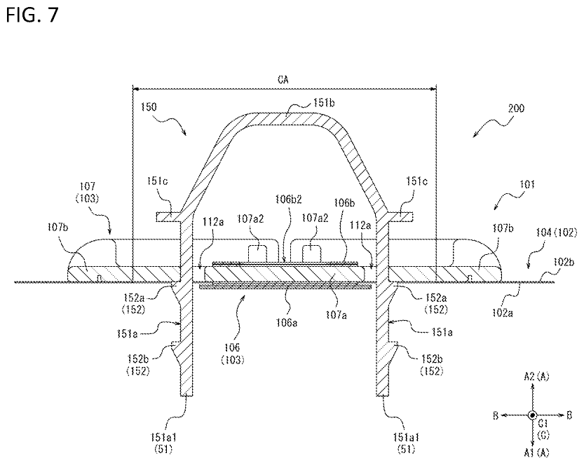

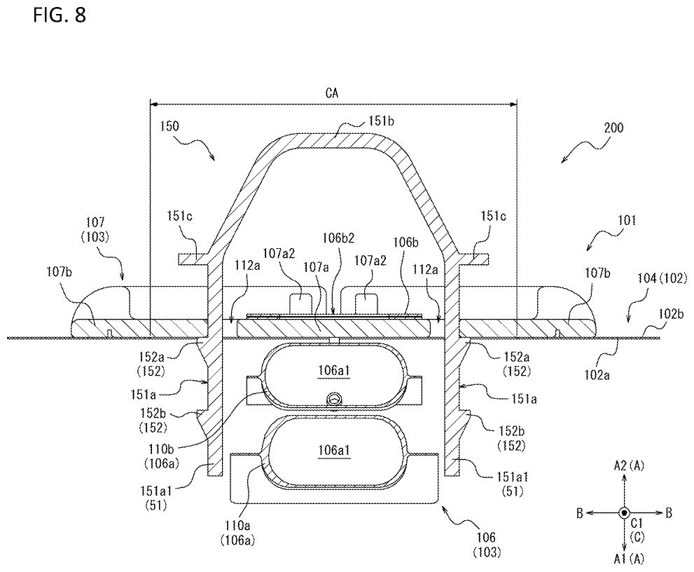

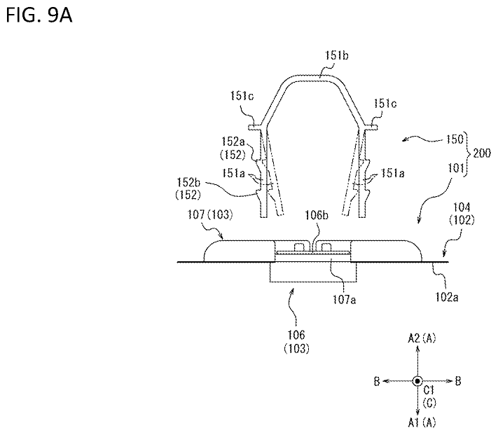

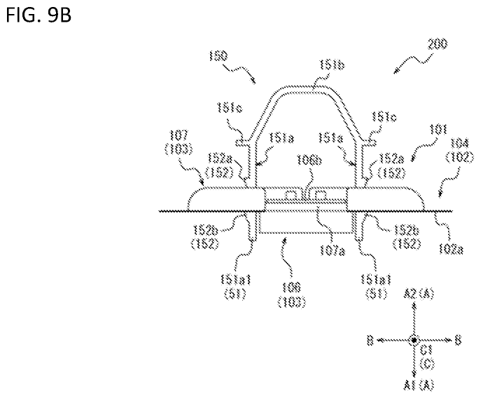

Next, with reference to FIGS. 4 to 9, a compression device set 200 according to a second embodiment of the present disclosure will be described. FIG. 4 is a perspective view of the compression device set 200 as viewed from the upper surface side. FIG. 5A is a top view of the compression device set 200. FIG. 5B is a bottom view of the compression device set 200. In FIGS. 5A and 5B, for convenience of description, a position of the blood vessel BV in the living body when a compression device 101 is attached on the biological surface is indicated by a dashed-two dotted line. FIG. 6 is a side view of the compression device set 200. FIG. 7 is a cross-sectional view of the compression device set 200 at a position taken along a line I-I in FIGS. 5A and 5B. FIG. 8 is a cross-sectional view of the compression device set 200 at the same position as in FIG. 7 in a state in which an inflatable portion serving as a compression portion 106a of the compression device 101 of the compression device set 200 is inflated. FIG. 9A is a view showing a state in which a pre-compression member 150 of the compression device set 200 is removed from the compression device 101. FIG. 9B is a view showing a state in which the pre-compression member 150 is fixed to the compression device 101 at a position different from that in FIGS. 4 to 8.

As shown in FIG. 4, the compression device set 200 includes the compression device 101 and the pre-compression member 150.

The compression device 101 can be adhered to the biological surface. The compression device 101 can compress the biological surface in a state of being adhered to the biological surface.

Specifically, the compression device 101 has an adhesion surface 102a that can be adhered to the biological surface. The compression device 101 can be attached to the biological surface by the adhesion surface 102a being adhered to the biological surface. As shown in FIG. 8, the compression device 101 includes the compression portion 106a that compresses the biological surface in the compression region CA in a state in which the adhesion surface 102a is adhered to the biological surface.

Hereinafter, among plan views of the compression device set 200 viewed along the direction orthogonal to the adhesion surface 102a of the compression device 101, a plan view (see FIG. 5A) viewed in the downward direction A1 from an upper side is simply referred to as a “top view” for convenience of description. Among the plan views of the compression device set 200 viewed along the direction orthogonal to the adhesion surface 102a of the compression device 101, a plan view (see FIG. 5B) viewed in the upward direction A2 from a lower side is simply referred to as a “bottom view” for convenience of description. When the top view and the bottom view are not distinguished from each other, the above views may be simply referred to as the “plan view”. Further, unless otherwise specified, the descriptions of the “plan view”, the “top view”, and the “bottom view” mean a plan view, a top view, and a bottom view when an inflatable portion serving as the compression portion 106a which will be described later is in a deflated form before inflation.

The compression portion 106a according to the present embodiment can compress the biological surface at a position sandwiched by the adhesion surface 102a and a position surrounded by the adhesion surface 102a in the plan views (see FIGS. 5A and 5B).

As shown in FIGS. 4 to 9, the pre-compression member 150 is attached to the compression device 101. As shown by a dashed-two dotted line in FIG. 9A, the pre-compression member 150 according to the present embodiment is attachable to and detachable from the compression device 101 by changing a shape of the pre-compression member 150. Specifically, the pre-compression member 150 according to the present embodiment can be detached from the compression device 101 and attached to the compression device 101 by elastically deforming as shown by the dashed-two dotted line in FIG. 9A. However, the pre-compression member 150 may not be detachable from the compression device 1.

The pre-compression member 150 can compress the biological surface to which the compression device 101 is to be adhered before the compression device 101 is adhered. More specifically, the pre-compression member 150 can include, in the compression region CA, the protruding portions 51 that protrude downward from the adhesion surface 102a of the compression device 1 and that can compress the biological surface before the adhesion surface 102a is adhered to the biological surface. The protruding portions 51 according to the present embodiment can compress the biological surface at the position between the compression portion 106a and the adhesion surface 102a in the compression region CA in the plan views (see FIGS. 5A and 5B).

The adhering method for adhering the compression device 101 to the biological surface by the compression device set 200 is the same as that of the compression device set 100 (see FIG. 1) according to the first embodiment described above, and thus description of the adhering method for adhering the compression device 101 to the biological surface is omitted here (see FIGS. 3A to 3E).

Hereinafter, further details of the compression device set 200 according to the present embodiment will be described.

Compression Device 101

As shown in FIGS. 4 to 9, the compression device 101 according to the present embodiment includes an adhesion body 102 and a compression member 103. The adhesion body 102 has the adhesion surface 102a described above that can be adhered to the biological surface. The compression member 103 is fixed to the adhesion body 102. The compression member 103 includes the compression portion 106a that can compress the biological surface in the state in which the adhesion surface 102a of the adhesion body 102 is adhered to the biological surface.

The adhesion body 102 according to the present embodiment is an adhesive sheet 104 having an adhesion surface 102a that can be adhered to the biological surface on a lower surface on one side in a thickness direction A. In the present embodiment, the “direction orthogonal to the adhesion surface 102a” described above is a direction that is the same as the thickness direction A of the adhesive sheet 104. The adhesion surface 102a is covered with a liner such as a release sheet in a state before use before being adhered to the biological surface. The liner is peeled and removed, for example, immediately before the adhesion surface 102a is adhered to the biological surface. The adhesive sheet 104 serving as the adhesion body 102 shown in FIGS. 4 to 9 shows a use state in which the liner is removed and the adhesion surface 102a is exposed.

The compression member 103 according to the present embodiment is fixed to the adhesive sheet 104. Specifically, the compression member 103 according to the present embodiment is fixed to an upper surface of the adhesive sheet 104, which is a surface opposite to the adhesion surface 102a. Hereinafter, a surface opposite to the adhesion surface 102a of the adhesive sheet 104 may be referred to as a “fixing surface 102b”. The compression member 103 includes the compression portion 106a that can compress the biological surface at a position different from the position at which the adhesion surface 102a is adhered in the state in which the adhesion surface 102a is adhered to the biological surface.

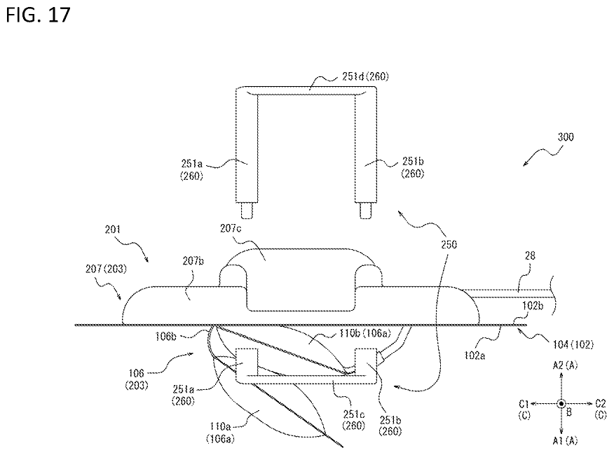

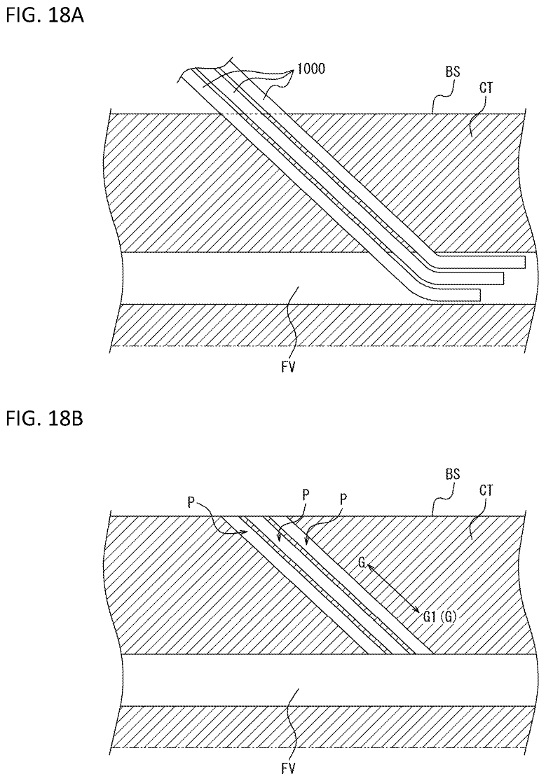

Accordingly, the compression device 101 is fixed to a position on the biological surface by adhering the adhesion surface 102a on the biological surface. According to the compression device 101, a predetermined site on the biological surface can be compressed by the compression portion 106a in the state in which the adhesion surface 102a is adhered to the biological surface. The predetermined site on the biological surface can include, for example, a wound on the biological surface or the vicinity of the wound formed by inserting a medical insertion member such as a puncture needle, a catheter, and a sheath into a blood vessel of a living body. After the medical insertion member is removed from the living body, the wound or the vicinity of the wound on the biological surface is compressed by the compression portion 106a, whereby a subcutaneous blood vessel BV (see FIGS. 5A and 5B) or perforations P (see FIG. 18B) extending from the blood vessel BV to the biological surface can be compressed from the biological surface. The compression portion 106a can perform compression for a predetermined time to stop bleeding.

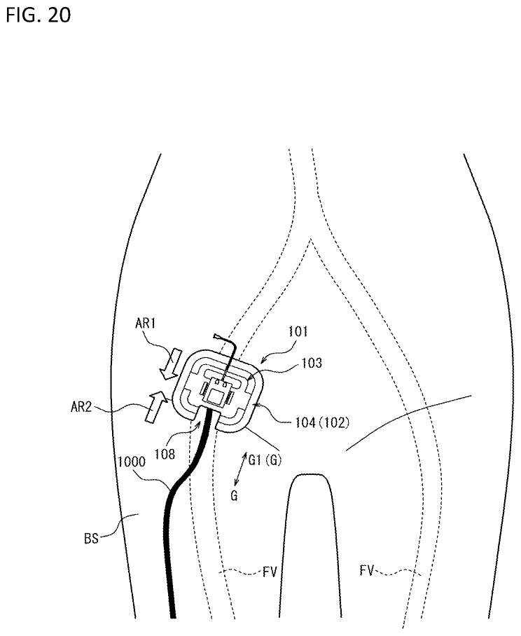

Hereinafter, each member and each portion of the compression device 101 according to the present embodiment will be described in detail. In the present embodiment, for convenience of description, one side of an extending direction C of the blood vessel BV described above is defined as a forward direction C1, and the other side of the extending direction C of the blood vessel BV is defined as a backward direction C2. The forward direction C1 means a side of the compression device 101 that receives a medical insertion member 1000 (see FIGS. 18A and 20) such as a sheath. The forward direction C1 according to the present embodiment refers to a side on which a receiving portion 108 is provided in the compression device 101. The backward direction C2 is a direction opposite to the forward direction C1 among the extending directions C. That is, the backward direction C2 according to the present embodiment refers to a side opposite to the side on which the receiving portion 108 is provided in the compression device 101.

Adhesion Body 102

As described above, the adhesion body 102 according to the present embodiment is the adhesive sheet 104. The adhesive sheet 104 has flexibility. Therefore, the adhesive sheet 104 can be deformed according to a shape of the biological surface. In addition, the adhesion surface 102a is likely to follow deformation of the biological surface. As a result, it is possible to prevent the compression device 101 from being unintentionally released from the biological surface.

The adhesion surface 102a of the adhesive sheet 104 according to the present embodiment is an entire lower surface of the adhesive sheet 104. The adhesion surface 102a of the adhesive sheet 104 may be provided only in a partial region of the lower surface of the adhesive sheet 104.

The adhesive sheet 104 includes a plurality of layers including, for example, a base material layer and an adhesive layer.

The base material layer can be formed of, for example, a thin resin sheet. More specifically, the base material layer can be formed of, for example, a white spunlace nonwoven fabric (i.e., nonwoven fabric) of polyester fibers, and has a thickness in a range of 5 μm to 150 μm, for example, 30 μm. However, a material of the base material layer is not limited to polyester, and may be, for example, an acrylic polymer, polyethylene, an ethylene-vinyl acetate copolymer, polyurethane, and a polyamide derivative.

The adhesive layer can be formed of, for example, an adhesive such as a rubber-based adhesive, an acrylic-based adhesive, and a silicon-based adhesive. The adhesive layer is stacked on the base material layer directly or indirectly with another layer interposed between the adhesive layer and the base material layer. The adhesion surface 102a of the adhesive sheet 104 according to the present embodiment is formed of the adhesive layer.

The adhesive sheet 104 may further include another layer in addition to the base material layer and the adhesive layer described above. The adhesive sheet 104 may include, for example, a surface layer. The surface layer can be formed of, for example, a resin having a thickness of about 5 μm to 50 μm. More specifically, examples of a material of the surface layer include polyester, polyamide, polyamideimide, polyethylene, polypropylene, polycarbonate, polyurethane, polyvinyl chloride, and a fluororesin. The surface layer is stacked on the base material layer directly or indirectly with another layer interposed between the surface layer and the base material layer on a side opposite to the adhesive layer with the base material layer interposed between the surface layer and the adhesive layer. Therefore, the upper surface of the adhesive sheet 104 may be a surface layer.

More specifically, the adhesive sheet 104 may be formed of a nonwoven fabric tape having an adhesive agent as an adhesive on one surface of the adhesive sheet 104. Further, the adhesive sheet 104 may be formed of a double-sided tape in which adhesive layers are provided on both sides of the base material layer. When the adhesive sheet 104 is formed of the double-sided tape, the compression member 103 can be fixed to the adhesive sheet 104 by adhering the compression member 103 to one adhesive layer of the adhesive sheet 104.

The adhesive sheet 104 according to the present embodiment has a substantially C-shaped outer shape in the plan views (see FIGS. 5A and 5B). As shown in FIGS. 5A and 5B, only a part of a lower surface side of the compression member 103 is covered with the adhesive sheet 104 according to the present embodiment. Specifically, only an outer edge region of a lower surface of the compression member 103 is covered with the adhesive sheet 104 according to the present embodiment. The adhesive sheet 104 according to the present embodiment is fixed only to the outer edge region of the lower surface of the compression member 103. In other words, the central region on the lower surface of the compression member 103 in which the inflatable portion serving as the compression portion 106a of the compression member 103 is located is not covered with the adhesive sheet 104 according to the present embodiment. The entire outer edge region of the lower surface of the compression member 103 is not covered with the adhesive sheet 104 according to the present embodiment, and a part of the outer edge region is covered. That is, a part of the outer edge region of the lower surface of the compression member 103 is not covered with the adhesive sheet 104 according to the present embodiment. According to the present embodiment, a portion of the outer edge region of the lower surface of the compression member 103 which is not covered with the adhesive sheet 104 is a portion adjacent to the receiving portion 108 in the compression member 103. The receiving portion 108 is a portion capable of receiving the medical insertion member 1000 (see FIGS. 18A and 20) to be inserted or already inserted into the blood vessel BV (see FIGS. 5A and 5B) of the living body. The receiving portion 108 according to the present embodiment is a gap defined by both ends of the adhesive sheet 104 having a substantially C-shape in the plan view.

Here, as shown in FIGS. 5A and 5B, the extending direction C according to the present embodiment are substantially the same as a front-rear direction of the compression device set 200 in the plan view. The front-rear direction is a linear direction passing through one end side of an outer edge of the compression device 101 on which the receiving portion 108 is provided and the other end side of the outer edge of the compression device 101 on a side opposite to the position at which the receiving portion 108 is provided. Hereinafter, a lateral direction orthogonal to the front-rear direction of the compression device set 200 in the plan view is referred to as a “width direction B of the compression device set 200” or a “width direction B”.

Compression Member 103

The compression member 103 according to the present embodiment includes the compression portion 106a and a support portion 107a. More specifically, the compression member 103 according to the present embodiment includes a compression body 106 and a support body 107. The compression portion 106a according to the present embodiment is formed of a portion of the compression body 106. The support portion 107a according to the present embodiment is formed of a part of the support body 107. Hereinafter, the compression body 106 and the support body 107 according to the present embodiment will be described in detail.

The compression body 106 according to the present embodiment includes the compression portion 106a and an extending portion 106b.

The compression portion 106a can press the biological surface in the state in which the adhesion surface 102a is adhered to the biological surface. Specifically, the compression portion 106a according to the present embodiment can press the biological surface by extending in the thickness direction A orthogonal to the adhesion surface 102a in the state in which the adhesion surface 102a is adhered to the biological surface. The compression portion 106a according to the present embodiment can press the biological surface in the compression region CA. The compression region CA is a position at which the adhesive sheet 104 is not present in the plan view. That is, the compression portion 106a according to the present embodiment can press the biological surface without interposing the adhesive sheet 104.

As shown in FIG. 8, the compression portion 106a according to the present embodiment is formed of the inflatable portion that defines accommodation spaces 106a1 capable of accommodating a fluid such as a gas in the accommodation spaces 106a1. More specifically, the inflatable portion serving as the compression portion 106a according to the present embodiment includes two balloon portions 110a and 110b that are connected to each other such that the inside of the balloon portion 110a and the inside of the balloon portion 110b communicate with each other. The accommodation spaces 106a1 according to the present embodiment are internal spaces in which the two balloon portions 110a and 110b communicate with each other.

The inflatable portion serving as the compression portion 106a can be inflated toward the downward direction A1 in the thickness direction A by supplying the fluid to the accommodation spaces 106a1. FIG. 7 shows a case of a deflated form before the inflatable portion serving as the compression portion 106a is inflated, and FIG. 8 shows a case of an inflated form after the inflatable portion serving as the compression portion 106a is inflated from the deflated form. The inflatable portion serving as the compression portion 106a according to the present embodiment is inflated toward the downward direction A1 by changing from the deflated form (see FIG. 7) to the inflated form (see FIG. 8), and is in a posture (or position) capable of compressing the biological surface. More specifically, the fluid is supplied to the accommodation spaces 106a1, so that the inflatable portion serving as the compression portion 106a receives a reaction force from a lower surface of the support portion 107a of the support body 107 and is inflated toward the downward direction A1. The fluid supplied to the accommodation spaces 106a1 of the inflatable portion serving as the compression portion 106a is not limited to the gas, and may be, for example, a liquid.

As shown in FIG. 7, the compression portion 106a in the deflated form is disposed along the lower surface of the support portion 107a of the support body 107 in a state in which the accommodation spaces 106a1 are deflated. The accommodation spaces 106a1 of the compression portion 106a communicates with a tube 28 extending to the outside of the support body 107. A fluid, for example, such as air is supplied through the tube 28 to the accommodation spaces 106a1 of the compression portion 106a from a fluid supply device connected to an inflation port serving as a connection portion 29 provided at an end portion of the tube 28. Accordingly, an inflated state of the compression portion 106a can be changed, and the compression portion 106a can be changed from the deflated form (see FIG. 7) to the inflated form (see FIG. 8).

The compression portion 106a according to the present embodiment is disposed between the biological surface and the lower surface of the support portion 107a in a state (hereinafter, may be described as an “adhering state of the compression device 101”) in which the adhesion surface 102a of the adhesive sheet 104 serving as the adhesion body 102 is adhered to the biological surface. When the compression portion 106a is inflated in the thickness direction A by the supply of the fluid in the adhered state of the compression device 101, the compression portion 106a is sandwiched between the biological surface and the support portion 107a and presses the biological surface. In other words, the compression portion 106a in the deflated form according to the present embodiment does not compress the biological surface in the adhered state of the compression device 101. However, the compression portion 106a may compress the predetermined site on the biological surface with a desired compression force by changing the form from the deflated form to the inflated form in the adhered state of the compression device 101. That is, the compression portion 106a in the deflated form may compress the biological surface in the adhered state of the compression device 101.

The extending portion 106b extends from the compression portion 106a in a sheet shape. The extending portion 106b has flexibility. The extending portion 106b is wound around the support portion 107a. Accordingly, the extending portion 106b extends from the compression portion 106a to an upper surface side of the support portion 107a on a side opposite to the compression portion 106a with the support portion 107a interposed between the extending portion 106b and the compression portion 106a. The extending portion 106b is locked to the support portion 107a on the upper surface side of the support portion 107a.

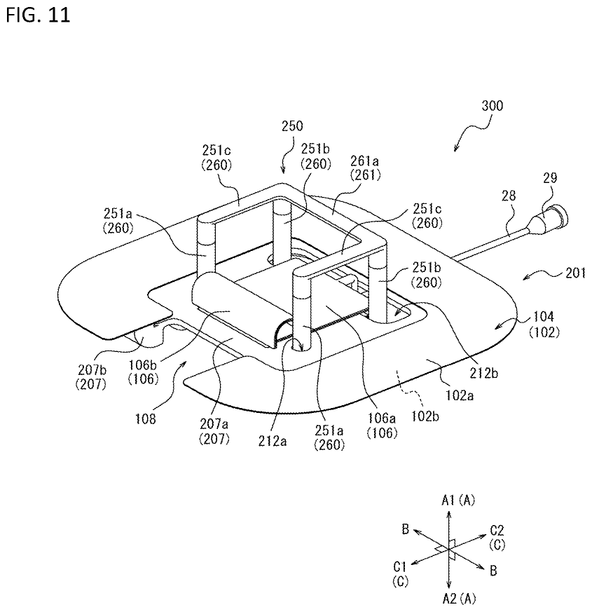

Specifically, the support portion 107a according to the present embodiment defines a through hole 107a1 penetrating in the thickness direction A. The support portion 107a according to the present embodiment includes locking protrusions 107a2 protruding in the upward direction A2. The extending portion 106b according to the present embodiment is wound around the support portion 107a through the through hole 107a1. More specifically, the extending portion 106b according to the present embodiment extends through the through hole 107a1 from the lower side at which the compression portion 106a is located with the support portion 107a interposed between the extending portion 106b and the compression portion 106a to the upper side which is an opposite side of the support portion 107a. The extending portion 106b according to the present embodiment is wound around the support portion 107a along an inner surface of the support portion 107a that defines the through hole 107a1 and the upper surface of the support portion 107a. In addition, a locking hole 106b1, into which the locking protrusion 107a2 is fitted on the upper surface side of the support portion 107a, is formed in the extending portion 106b. By fitting the locking protrusion 107a2 into the locking hole 106b1, the extending portion 106b is positioned on the support portion 107a. In addition, the extending portion 106b is wound around from a lower surface side to the upper surface side of the support portion 107a at a position on a receiving portion 108 side with respect to the compression portion 106a. That is, the through hole 107a1 according to the present embodiment is located on the receiving portion 108 side with respect to the compression portion 106a. Therefore, the compression portion 106a and the extending portion 106b that constitute the compression body 106 according to the present embodiment are curved in a substantially U-shape as a whole. Accordingly, the inflatable portion serving as the compression portion 106a can be inflated while pivoting about the portion of the extending portion 106b connected to the compression portion 106a as a hinge portion. A configuration of the compression body 106 according to the present embodiment is similar to that of a compression device set 300 (see FIGS. 10 to 17) according to a third embodiment to be described later.

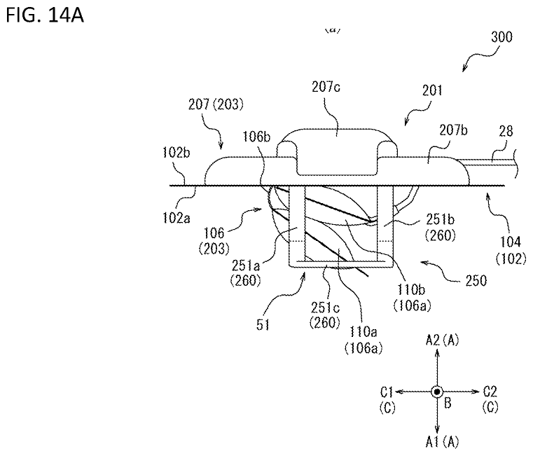

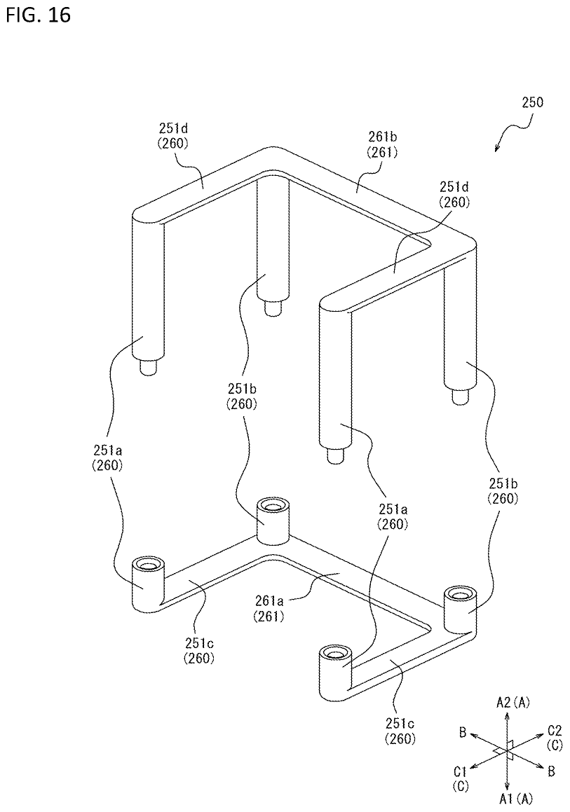

The inflatable portion serving as the compression portion 106a can be inflated not only toward the thickness direction A but also toward a direction inclined with respect to the thickness direction A by being pivoted by a hinge portion (see the compression body 106 in FIGS. 14 and 17 showing the third embodiment) of the extending portion 106b. As described above, the compression body 106 according to the present embodiment is fixed to the support portion 107a in a state in which the extending portion 106b having the sheet shape is wound around the upper and lower surfaces of the support portion 107a through the through hole 107a1 of the support portion 107a of the support body 107. Therefore, at the time of inflating, the inflatable portion serving as the compression portion 106a is inflated while pivoting about a pivot center with the hinge portion, which is a portion of the extending portion 106b connected to the compression portion 106a on a lower side of the through hole 107a1, as the pivot center (see FIGS. 14 and 17 showing the third embodiment).

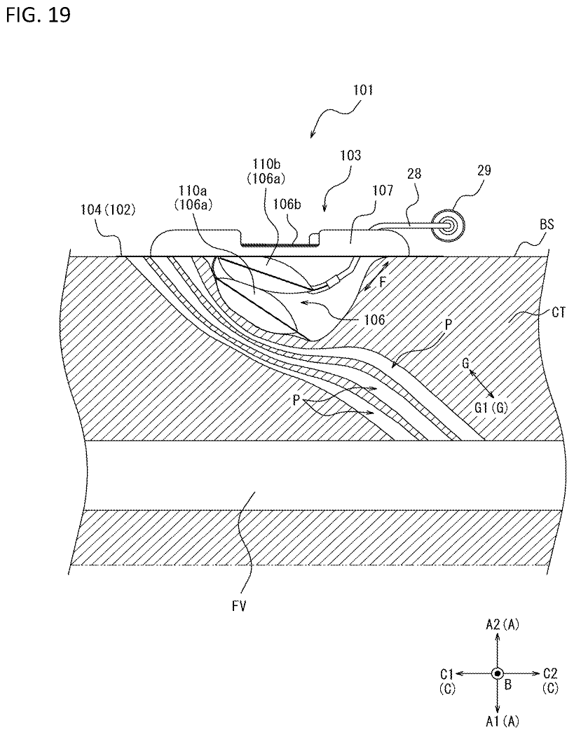

More specifically, the two balloon portions 110a and 110b (see FIG. 8) constituting the inflatable portion serving as the compression portion 106a according to the present embodiment overlap with each other in the thickness direction A. One end of each of the two balloon portions 110a and 110b is attached to the extending portion 106b. That is, one end side (left side in FIGS. 14 and 17) of each of the two balloon portions 110a and 110b is restrained by the extending portion 106b. Therefore, even when the two balloon portions 110a and 110b are inflated, the two balloon portions 110a and 110b are not separated from each other on the one end side described above. On the other hand, the other end side (right side in FIGS. 14 and 17) of each of the two balloon portions 110a and 110b is not restrained at all. Therefore, when the two balloon portions 110a and 110b are inflated, the two balloon portions 110a and 110b are separated from each other on the other end side described above. That is, in the two balloon portions 110a and 110b constituting the compression portion 106a according to the present embodiment, the other end side which is not attached to the extending portion 106b pivots about a pivot center with the one end side attached to the extending portion 106b as the pivot center. In this way, the inflatable portion serving as the compression portion 106a according to the present embodiment is inflated toward the direction inclined with respect to the thickness direction A. By inflating the inflatable portion toward the direction inclined with respect to the thickness direction A, the perforations P (see FIG. 18B) to be described later are likely to be narrowed or obstructed. The details will be described later (see FIG. 19). However, a configuration for inflating the inflatable portion toward the direction inclined with respect to the thickness direction A is not limited to a configuration of the compression body 106 according to the present embodiment.

The compression body 106 according to the present embodiment is formed of a member having translucency, and can be viewed in the direction orthogonal to the adhesion surface 102a. The same applies to at least the support portion 107a of the support body 107 to be described later. Therefore, according to the compression device 101 in the present embodiment, the compression position on the biological surface can be visually recognized through the compression portion 106a and the extending portion 106b of the compression body 106 and the support portion 107a of the support body 107. However, as in the extending portion 106b according to the present embodiment, a visual recognition through hole 106b2 may be formed to more easily and visually recognize the compression position on the biological surface.

As constituent materials of the compression portion 106a and the extending portion 106b of the compression body 106, for example, soft polyvinyl chloride, polyurethane, polyethylene, polypropylene, polyester, ethylene-vinyl acetate copolymer (EVA), silicone, or a material having flexibility and obtained by mixing any of these materials can be used.

The support body 107 according to the present embodiment includes the support portion 107a and a fixing portion 107b. The fixing portion 107b is a portion overlapping the adhesive sheet 104 serving as the adhesion body 102 in the plan views (see FIGS. 5A and 5B). The fixing portion 107b is fixed to the adhesive sheet 104 serving as the adhesion body 102 at the fixing surface 102b located on a back side of the adhesion surface 102a. The support portion 107a supports the inflatable portion constituting the compression portion 106a. The support portion 107a is a portion that does not overlap the adhesive sheet 104 serving as the adhesion body 102 in the plan views (see FIGS. 5A and 5B). Further, the support portion 107a overlaps the compression portion 106a in the plan views (see FIGS. 5A and 5B).

In other words, the fixing portion 107b according to the present embodiment is implemented by an outer edge portion of the support body 107 in the plan view. In addition, the support portion 107a according to the present embodiment is implemented by a central portion of the support body 107 in the plan view.

The fixing portion 107b according to the present embodiment extends in a substantially C-shape such that a substantially entire region thereof overlaps the adhesive sheet 104 in the plan view. Both ends of each of the adhesive sheet 104 and the fixing portion 107b that extend in the substantially C-shape in the plan view define a gap between the ends of each of the adhesive sheet and the fixing portion 107b. The gap constitutes the receiving portion 108 of the compression device 101 capable of receiving the medical insertion member 1000 (see FIGS. 18A and 20) to be inserted or already inserted into the blood vessel BV (see FIGS. 5A and 5B) of the living body.

The receiving portion 108 according to the present embodiment is implemented by the gap between both ends of each of the adhesive sheet 104 and the fixing portion 107b, and a configuration of the receiving portion 108 is not particularly limited. The receiving portion 108 may be implemented by a concave portion formed at the outer edge of the compression device 101 in the plan view.

As described above, the support portion 107a according to the present embodiment includes a portion that overlaps the compression portion 106a in the plan view and that supports the compression portion 106a. The through hole 107a1 described above is formed in the support portion 107a. The support portion 107a includes the locking protrusion 107a2 described above that is fitted into the locking hole 106b1 in the extending portion 106b of the compression body 106. The compression portion 106a of the compression body 106 described above is disposed on the lower surface side of the support portion 107a.

Accordingly, in the support body 107 according to the present embodiment, only the lower surface of the fixing portion 107b is fixed to the fixing surface 102b of the adhesive sheet 104 having a substantially C-shape in the plan view. In other words, the lower surface of the support portion 107a is not covered with the adhesive sheet 104. Therefore, the lower surface of the support portion 107a is not fixed to the fixing surface 102b of the adhesive sheet 104.

The compression portion 106a in the inflated form is sandwiched between the support portion 107a and the biological surface. Specifically, the support portion 107a according to the present embodiment includes a portion located on an upper side with respect to the inflatable portion serving as the compression portion 106a. Therefore, when the compression portion 106a inflates in the state in which the adhesion surface 102a of the adhesion body 102 is adhered to the biological surface, the compression portion 106a is vertically sandwiched between the support portion 107a and the biological surface. Accordingly, the biological surface is compressed by the compression portion 106a.

The support body 107 according to the present embodiment is flat in the thickness direction A and has a substantially quadrangular outer shape in the plan view, and the shape of the support body 107 is not particularly limited. In the support body 107 according to the present embodiment, a maximum thickness in the thickness direction A is larger in the fixing portion 107b at the outer edge portion than in the support portion 107a at the central portion, and a thickness relation of the maximum thickness in the thickness direction A that is larger in the fixing portion 107b at the outer edge portion than in the support portion 107a at the central portion is not particularly limited.

Here, as shown in FIGS. 4, 5, 7, and 8, the support portion 107a defines lateral through holes 112a and a rear through hole 112b that penetrate in the thickness direction A orthogonal to the adhesion surface 102a.

The lateral through holes 112a are formed on both sides in the width direction B with the compression portion 106a interposed between the lateral through holes 112a in the plan view. The lateral through hole 112a is formed at a position sandwiched between the compression portion 106a and the adhesion surface 102a in the width direction B in the plan view (see FIG. 5B). Details will be described later, and the pre-compression member 150 according to the present embodiment can be attached to the compression device 101 in a state of passing through the lateral through holes 112a.

The rear through hole 112b is formed at a position in the backward direction C2 in the extending direction C with respect to the compression portion 106a in the plan view. The rear through hole 112b is formed at a position sandwiched between the compression portion 106a and the adhesion surface 102a in the extension direction C in the plan view.

Examples of a material of the support body 107 according to the present embodiment include a resin material. Examples of the resin material include thermoplastic resins used in injection molding such as an ABS resin, an AS resin, polyethylene, polypropylene, polystyrene, polyvinyl chloride, a polyvinylidene chloride resin, polyphenylene oxide, thermoplastic polyurethane, polymethylene methacrylate, polyoxyethylene, a fluororesin, polycarbonate, polyamide, an acetal resin, an acrylic resin, and polyethylene terephthalate, and thermosetting resins such as a phenol resin, an epoxy resin, a silicone resin, and unsaturated polyester.

Pre-Compression Member 150

The pre-compression member 150 according to the present embodiment has a substantially U-shaped outer shape. Regarding the pre-compression member 150 according to the present embodiment, two tip portions 151a1 of the substantial U-shape serving as the protruding portions 51 are inserted into the lateral through holes 112a in the support portion 107a of the compression device 101 from the upper side in the downward direction A1. Accordingly, the pre-compression member 150 can protrude downward from the adhesion surface 102a of the compression device 101. That is, the protruding portions 51 of the pre-compression member 150 according to the present embodiment protrude downward from the adhesion surface 102a through the lateral through holes 112a serving as the through holes.

Specifically, the pre-compression member 150 according to the present embodiment can include two elongated plate-shaped insertion portions 151a that are inserted into the lateral through holes 112a in the support portion 107a of the compression device 101, and an interlock portion 151b that interlocks one end portion of each of the two insertion portions 151a in a longitudinal direction.

The two insertion portions 151a face each other in the width direction B. The interlock portion 151b interlocks the two insertion portions 151a in a state in which the two insertion portions 151a are spaced apart in the width direction B.

The two insertion portions 151a are elastically deformable in a manner of approaching each other in the width direction B, which is a facing direction, with the interlock portion 151b as a fulcrum.

Convex portions 152 protruding outward in the width direction B are provided on outer surfaces of the two insertion portions 151a opposite to inner surfaces of the two insertion portions 151a. The inner surfaces face each other. Each of the insertion portions 151a according to the present embodiment includes a plurality of (two in the present embodiment) convex portions 152 at different positions in the thickness direction A. Specifically, each of the two insertion portions 151a includes an upper convex portion 152a and a lower convex portion 152b. The upper convex portion 152a of the one insertion portion 151a and the upper convex portion 152a of the other insertion portion 151a are provided at the same position in the thickness direction A. The lower convex portion 152b of the one insertion portion 151a and the lower convex portion 152b of the other insertion portion 151a are provided at the same position in the thickness direction A.

The pre-compression member 150 according to the present embodiment further includes grasping units 151c that protrude outward in the width direction B at positions at which the insertion portions 151a and the interlock portion 151b are connected to each other. The grasping units 151c are located above the convex portions 152 of the insertion portions 151a.

As shown in FIGS. 7 and 8, the two insertion portions 151a according to the present embodiment can be inserted into the lateral through holes 112a in the support portion 107a of the compression device 101. The convex portions 152 of each of the insertion portions 151a can pass through the lateral through holes 112a by the two insertion portions 151a being elastically deformed in a manner of approaching each other (see the dashed-two dotted line in FIG. 9A). The insertion portions 151a according to the present embodiment restrict the convex portions 152 from being unintentionally pulled out from the lateral through holes 112a in the upward direction A2 by being caught by lower edge portions of the lateral through holes 112a. When the insertion portions 151a are pulled out from the lateral through holes 112a in the upward direction A2, the two insertion portions 151a can be elastically deformed in a manner of approaching each other (see the dashed-two dotted line in FIG. 9A). Accordingly, the convex portion 152 does not interfere with the edge portion of the lateral through hole 112a. Therefore, the insertion portion 151a can be pulled out from the lateral through hole 112a in the upward direction A2.

As described above, the insertion portion 151a according to the present embodiment includes the upper convex portion 152a and the lower convex portion 152b. FIGS. 4 to 8 show a state in which the upper convex portion 152a of the insertion portion 151a is caught on the lower surface of the support portion 107a of the compression device 101. On the other hand, FIG. 9B shows a state in which the lower convex portion 152b of the insertion portion 151a is caught on the lower surface of the support portion 107a of the compression device 101. Accordingly, in the compression device set 200 according to the present embodiment, the attachment position of the pre-compression member 150 in the thickness direction A with respect to the compression device 101 can be changed.

Accordingly, in the pre-compression member 150 according to the present embodiment, the tip portions 151a1 on the lower side of the two insertion portions 151a protrude downward from the adhesion surface 102a of the compression device 101. That is, in the pre-compression member 150 according to the present embodiment, the protruding portions 51 that pre-compresses the biological surface are implemented by the tip portions 151a1 on the lower side of the two insertion portions 151a.

The grasping unit 151c is caught on the upper surface of the support portion 107a including the upper edge portion of the lateral through hole 112a, so that the pre-compression member 150 according to the present embodiment is restricted from falling out of (or disengaging from) the lateral through hole 112a in the downward direction A1. That is, the grasping units 151c are portions grasped by a user such as a health care worker to carry the compression device set 200 in a state in which the pre-compression member 150 is attached to the compression device 101, and is also a falling-off restricting portion that restricts the pre-compression member 150 from falling off (or disengaging) in the downward direction A1.

Accordingly, the pre-compression member 150 according to the present embodiment is attached to the compression device 101 in a manner of being relatively movable in the thickness direction A orthogonal to the adhesion surface 102a. Accordingly, it is possible to adjust a protrusion amount of the tip portions 151a1 serving as the protruding portions 51 on the lower side of the insertion portions 151a downward from the adhesion surface 102a.

Further, in the present embodiment, the convex portion 152 of the pre-compression member 150 is caught from the lower side on the lower edge portion of the lateral through-hole 112a in the compression device 101, thereby restricting the pre-compression member 150 from being pulled out upward from the compression device 101. Accordingly, a minimum protrusion amount of the tip portions 151a1 on the lower sides of the insertion portions 151a downward from the adhesion surface 102a is fixed.

In other words, the compression device 101 and the pre-compression member 150 according to the present embodiment include a movement restriction mechanism that fixes the minimum protrusion amount of the protruding portions 51. The movement restriction mechanism restricts movement of the pre-compression member 150 relative to the compression device 101 in the direction orthogonal to the adhesion surface 102a. Specifically, in the present embodiment, the lower edge portion of the lateral through hole 112a in the compression device 101 engages with the convex portion 152 of the pre-compression member 150 to restrict movement of the pre-compression member 150 relative to the compression device 101 in the upward direction A2. Accordingly, the minimum protrusion amount of the tip portion 151a1 of the insertion portion 151a is fixed. The tip portion 151a1 serves as the protruding portion 51.

The compression device set 200 includes such a movement restriction mechanism, so that a minimum pre-compression depth of the biological surface pre-compressed by the protruding portion 51 can be defined.

The movement restriction mechanism according to the present embodiment can adjust the minimum protrusion amount of the protruding portion 51 in a plurality of stages. Specifically, the pre-compression member 150 according to the present embodiment includes the upper convex portions 152a and the lower convex portions 152b provided at different positions in the thickness direction A. By catching the upper convex portion 152a on the lower edge portion of the lateral through hole 112a in the compression device 101, the minimum protrusion amount of the tip portion 151a1 of the insertion portion 151a can be increased as compared with a case in which the lower convex portion 152b is caught on the lower edge portion of the lateral through hole 112a (see FIGS. 6 and 9B, and the like). The tip portion 151a1 serves as the protruding portion 51. That is, the minimum protrusion amount of the protruding portion 51 can be adjusted in the plurality of stages (two stages in the present embodiment). A lower end of the protruding portion 51 according to the present embodiment is located at, in the direction orthogonal to the adhesion surface 102a, a position that is the same as or above a lower end of the compression portion 106a when the inflatable portion serving as the compression portion 106a is maximally inflated in a state in which the compression device 101 is not adhered to the biological surface. Accordingly, excessive pre-compression can be reduced.

In the present embodiment, the minimum protrusion amount of the protruding portion 51 can be adjusted, for example, in two stages, and may be adjusted in three or more stages.

The movement restriction mechanism according to the present embodiment includes the lower edge portion of the lateral through hole 112a of the compression device 101 and the convex portion 152 of the pre-compression member 150, and is not limited to the configuration. The movement restriction mechanism may be another mechanism as long as the mechanism fixes the minimum protrusion amount of the protruding portion 51.

Further, the pre-compression member 150 according to the present embodiment is attachable to and detachable from the compression device 101. Specifically, as described above, the pre-compression member 150 can be pulled out upward from the lateral through hole 112a by elastically deforming the two insertion portions 151a facing each other in a manner of approaching each other. Accordingly, the pre-compression member 150 can be detached from the compression device 101 after the compression device 101 is adhered to the biological surface. Therefore, after the compression device 101 is attached to the biological surface, the pre-compression member 150 can be prevented from remaining on the biological surface and becoming an obstacle.

The protruding portions 51 of the pre-compression member 150 according to the present embodiment are provided on both sides sandwiching the compression portion 106a of the compression device 101 in the plan view (see FIG. 5B). That is, the pre-compression member 150 according to the present embodiment includes, as the protruding portions 51, a first protruding portion and a second protruding portion that are provided on both sides sandwiching the compression portion 106a of the compression device 101 in the plan view (see FIG. 5B). In the present embodiment, the tip portions 151a1 of the two insertion portions 151a are provided on both sides sandwiching the compression portion 106a of the compression device 101 in the plan view. The tip portion 151a1 serves as the protruding portion 51. That is, the tip portion 151a1 of the one insertion portion 151a constitutes the first protruding portion. The tip portion 151a1 of the other insertion portion 151a constitutes the second protruding portion. Accordingly, it is possible to pre-compress the biological surface on both sides sandwiching the blood vessel that is compressed by the compression portion 106a of the compression device 101 and that is narrowed or obstructed, and it is possible to reduce interference between the medical insertion member 1000 (see FIGS. 18A and 20) such as a sheath inserted into the living body and the protruding portion 51.

Examples of a material of the pre-compression member 150 according to the present embodiment include the resin material exemplified as the material of the support body 107 of the compression device 101 described above.

### Third Embodiment

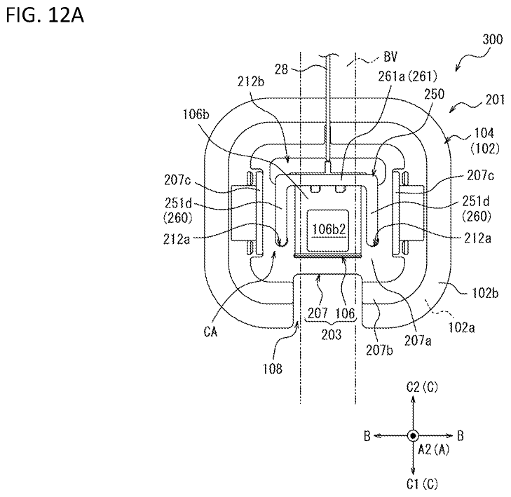

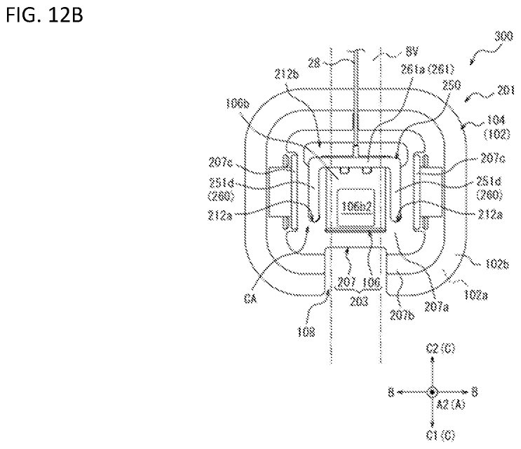

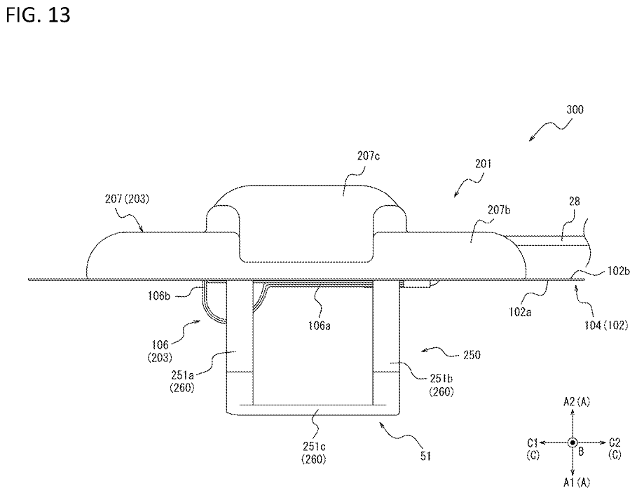

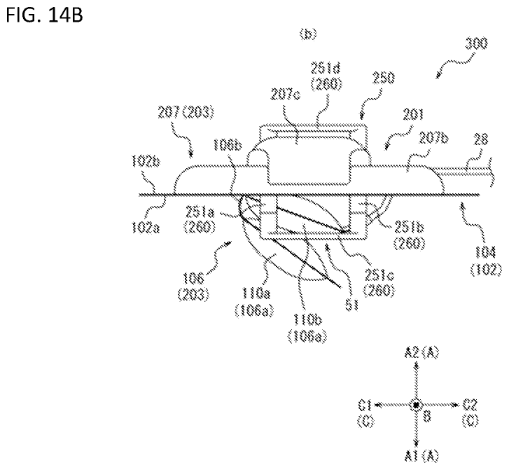

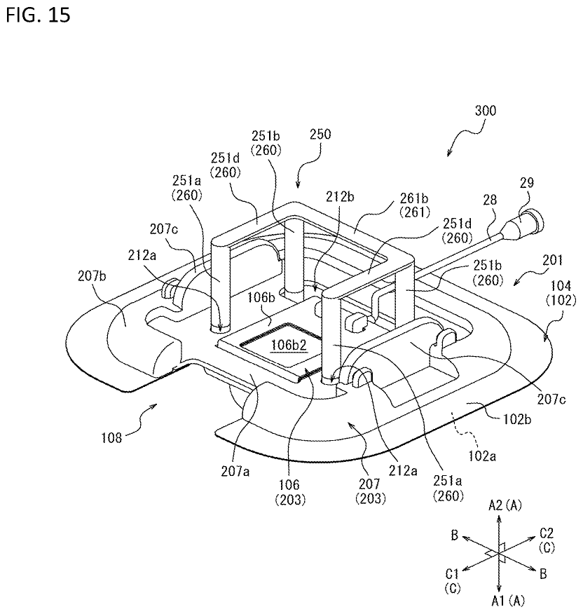

Next, with reference to FIGS. 10 to 17, the compression device set 300 according to a third embodiment of the present disclosure will be described. FIG. 10 is a perspective view of the compression device set 300 as viewed from the upper surface side. FIG. 11 is a perspective view of the compression device set 300 as viewed from the lower surface side. FIG. 12A is a top view of the compression device set 300. FIG. 12B is a bottom view of the compression device set 300. In FIGS. 12A and 12B, for convenience of description, a position of a blood vessel BV in a living body when a compression device 201 is attached on a biological surface is indicated by a dashed-two dotted line. FIG. 13 is a side view of the compression device set 300, and is a view showing a case in which an inflatable portion serving as the compression portion 106a is in a deflated form. FIG. 14A is a side view of the compression device set 300, and is a view showing a case in which the inflatable portion serving as the compression portion 106a is in an inflated form. FIG. 14B is a side view of the compression device set 300 in a state in which a position of a pre-compression member 250 is different from that in FIG. 14A. FIG. 15 is a perspective view of the compression device set 300 as viewed from the upper surface side, and shows a state in which the position of the pre-compression member 250 is different from that in FIG. 10. FIG. 16 is an exploded perspective view of the pre-compression member 250 alone. FIG. 17 is a side view of the compression device set 300 showing a state in which the pre-compression member 250 is removed from the compression device 201 in the compression device set 300.

The compression device set 300 according to the present embodiment differs from the compression device set 200 according to the second embodiment in a configuration of a support body 207 of the compression device 201 and a configuration of the pre-compression member 250. Here, these differences will be mainly described, and the description of a configuration common to the compression device set 200 according to the second embodiment will be omitted.

The compression device set 300 includes the compression device 201 and the pre-compression member 250.

The compression device 201 includes the adhesion body 102 and a compression member 203. The compression member 203 according to the present embodiment includes the compression body 106 and the support body 207.

The support body 207 according to the present embodiment includes a support portion 207a, a fixing portion 207b, and a grasping unit 207c.

As compared with the support portion 107a (see FIG. 4) according to the second embodiment, the support portion 207a is the same as the support portion 107a except for shapes of lateral insertion holes 212a and backward insertion holes 212b, and thus the description of support portion 207a is omitted here.

As compared with the fixing portion 107b (see FIG. 4) according to the second embodiment, the fixing portion 207b has substantially the same outer shape as the fixing portion 107b, and thus the description of the fixing portion 207b is omitted here.

The grasping unit 207c protrudes in the upward direction A2 at a position between the support portion 207a and the fixing portion 207b in a plan view (see FIG. 12A). The grasping unit 207c includes two grasping plate portions facing each other in the width direction B. A user such as a health care worker can carry the compression device set 300 or the compression device 201 alone by grasping the grasping unit 207c.

The pre-compression member 250 according to the present embodiment has an outer shape of a box-type frame shape. Specifically, the pre-compression member 250 according to the present embodiment includes two rectangular frame-shaped portions 260 that face each other at positions at which the two frame-shaped portions 260 are spaced apart in the width direction B, and an interlock portion 261 that interlocks the two frame-shaped portions 260 at end portions in the backward direction C2 of the extending direction C. End portions of the two frame-shaped portions 260 in the forward direction C1 of the extending direction C are not connected to each other.

As shown in FIGS. 10 and 11, each frame-shaped portion 260 includes a rod-shaped lateral insertion portion 251a inserted through the lateral insertion hole 212a of the compression device 201. Each frame-shaped portion 260 includes a rod-shaped backward insertion portion 251b inserted into the backward insertion hole 212b. Further, each frame-shaped portion 260 includes a rod-shaped lower frame portion 251c that interlocks a lower end of the lateral insertion portion 251a and a lower end of the backward insertion portion 251b. Furthermore, each frame-shaped portion 260 includes a rod-shaped upper frame portion 251d that interlocks an upper end of the lateral insertion portion 251a and an upper end of the backward insertion portion 251b. As shown in FIGS. 10 and 11, each frame-shaped portion 260 is attached to the compression device 201 in a state in which the lower frame portion 251c interlocks the lateral insertion portion 251a and the backward insertion portion 251b on a lower side of the support portion 207a and the upper frame portion 251d interlocks the lateral insertion portion 251a and the backward insertion portion 251b on an upper side of the support portion 207a. Therefore, the pre-compression member 250 is movable in the direction orthogonal to the adhesion surface 102a with respect to the compression device 201 from a position at which the upper frame portion 251d of the frame-shaped portion 260 abuts on an upper surface (in the present embodiment, an upper surface of a locking protrusion 207a2 that is a portion of an upper surface of the support portion 207a) of the support portion 207a to a position at which the lower frame portion 251c of the frame-shaped portion 260 abuts on a lower surface of the support portion 207a. FIGS. 10 to 14A show a state in which the upper frame portion 251d of the frame-shaped portion 260 abuts on the upper surface of the support portion 207a. FIG. 15 shows a state in which the lower frame portion 251c of the frame-shaped portion 260 abuts on the lower surface of the support portion 207a. FIG. 14B shows a state in which the upper frame portion 251d of the frame-shaped portion 260 does not abut on the upper surface of the support portion 207a and the lower frame portion 251c of the frame-shaped portion 260 does not abut on the lower surface of the support portion 207a.

The interlock portion 261 according to the present embodiment includes a rod-shaped lower side interlocking piece 261a that interlocks lower ends of the backward insertion portions 251b of the two frame-shaped portions 260. The interlock portion 261 according to the present embodiment includes a rod-shaped upper interlocking piece 261b that interlocks the upper ends of the backward insertion portions 251b of the two frame-shaped portions 260.

Accordingly, in the pre-compression member 250 according to the present embodiment, the two frame-shaped portions 260 that face each other in the width direction B are interlocked by the interlock portion 261 and are integrated. Therefore, in the pre-compression member 250, the two frame-shaped portions 260 and the interlock portion 261 are integrated and move relative to the compression device 201 in the thickness direction A.

As shown in FIG. 16, the pre-compression member 250 according to the present embodiment is separable into two parts. Specifically, in the pre-compression member 250 according to the present embodiment, the lateral insertion portion 251a and the backward insertion portion 251b are separable into two parts in the longitudinal direction. Each of the lateral insertion portions 251a and the backward insertion portions 251b according to the present embodiment is separated into two parts by pulling both end portions in the longitudinal direction outward in the longitudinal direction. The lateral insertion portions 251a according to the present embodiment are connected by fitting one part of two separable parts into the other part, and a connection unit of the lateral insertion portions 251a is not particularly limited. The same applies to the backward insertion portions 251b. That is, the backward insertion portions 251b according to the present embodiment are connected by fitting one part of two separable parts into the other part, and a connection unit of the backward insertion portions 251b is not particularly limited.

Next, an adhering method for adhering the compression device 201 to the biological surface using the compression device set 300 according to the present embodiment will be described.

First, FIGS. 10 to 13 show a state in which the pre-compression member 250 is located at a lowermost side with respect to the compression device 201, that is, a state in which the upper frame portion 251d of the frame-shaped portion 260 of the pre-compression member 250 abuts on the upper surface of the support portion 207a. In the state, the inflatable portion serving as the compression portion 106a is in the deflated form. As shown in FIGS. 10, 11, and 13, a lower end portion of the pre-compression member 250 protrudes downward from the adhesion surface 102a of the compression device 201. That is, in the present embodiment, the lower end portion of the pre-compression member 250 constitutes the protruding portion 51 that pre-compresses the biological surface. In the state, the adhesion surface 102a of the compression device 201 is moved in the downward direction A1 and approaches the biological surface. Therefore, before the adhesion surface 102a is adhered to the biological surface, the lower end portion constituting the protruding portion 51 of the pre-compression member 250 pre-compresses the biological surface. Specifically, the lower frame portion 251c of the frame-shaped portion 260 of the pre-compression member 250 and the lower side interlocking piece 261a of the interlock portion 261 come into contact with the biological surface to press the biological surface in the downward direction A1.

Then, the adhesion surface 102a of the compression device 201 is adhered to the biological surface in a state in which the biological surface is pre-compressed by the pre-compression member 250.

Here, a protrusion amount of the protruding portion 51 of the pre-compression member 250 downward from the adhesion surface 102a will be described later. FIG. 14A shows a state in which the pre-compression member 250 is located at a lowermost side with respect to the compression device 201, that is, the same state as in FIGS. 10 to 13. The state indicates a maximum protrusion amount of the protruding portion 51 of the pre-compression member 250 that most protrudes downward from the adhesion surface 102a. FIG. 14A shows a state in which the inflatable portion serving as the compression portion 106a is maximally inflated in a state in which the compression device 201 is not adhered to the biological surface. As shown in FIG. 14A, the lower end of the protruding portion 51 according to the present embodiment in the state in which the protruding portion 51 most protrudes downward from the adhesion surface 102a is located at a position that is substantially equal in the thickness direction A to a lower end of the inflatable portion serving as the compression portion 106a in the inflated form. In other words, the lower end of the protruding portion 51 according to the present embodiment is not located lower than the lower end of the inflatable portion serving as the compression portion 106a in the inflated form. Accordingly, excessive pre-compression performed by the protruding portion 51 can be reduced.

In the compression device set 300 according to the present embodiment, the pre-compression member 250 is movable in the thickness direction A with respect to the compression device 201. Therefore, as shown in FIG. 14B, pre-compression may be performed in a state in which the protrusion amount of the protruding portion 51 of the pre-compression member 250 is smaller than that in the state shown in FIG. 14A. The protrusion amount of the protruding portion 51 may be appropriately adjusted by the user such as the health care worker, for example, according to a hardness of a skin of a patient.

FIG. 15 shows a state in which the pre-compression performed by the pre-compression member 250 is released after the adhesion surface 102a of the compression device 201 is adhered to the biological surface. Specifically, FIG. 15 shows a state in which the pre-compression member 250 is located on an uppermost side with respect to the compression device 201, that is, a state in which the lower frame portion 251c (see FIG. 11) of the frame-shaped portion 260 abuts on the lower surface of the support portion 207a. In the state shown in FIG. 15, an upper end of the pre-compression member 250 is pulled up in the upward direction A2 while the compression device 201 is pressed on the biological surface. Accordingly, as shown in FIG. 17, in the pre-compression member 250, the lateral insertion portion 251a and the backward insertion portion 251b are each vertically separated into two parts. That is, the separated upper side portion of the pre-compression member 250 can be separated upward from the compression device 201 and removed from the compression device 201. On the other hand, the separated lower side portion of the pre-compression member 250 remains between the support portion 207a of the support body 207 of the compression device 201 and the biological surface. However, as shown in FIG. 17, by inflating the inflatable portion serving as the compression portion 106a, a gap is formed between the support portion 207a and the biological surface in the vicinity of the compression portion 106a. The separated lower side portion of the pre-compression member 250 is located in the gap and does not interfere with the compression performed by the compression portion 106a on the biological surface.

In particular, as shown in FIGS. 12A and 12B, the lower frame portion 251c of the frame-shaped portion 260 constituting the protruding portion 51 of the pre-compression member 250 is located between the adhesion surface 102a and the compression portion 106a in the compression region CA in plan views. The compression portion 106a according to the present embodiment inflates while pivoting, as described in the second embodiment. Therefore, the lower side interlocking piece 261a of the interlock portion 261 constituting the protruding portion 51 of the pre-compression member 250 is disposed at a position overlapping an outer edge portion of the compression portion 106a in the plan views (see FIGS. 12A and 12B), but does not interfere with inflation of the compression portion 106a. Accordingly, the protruding portion 51 of the pre-compression member 250 according to the present embodiment pre-compresses the biological surface at a position different from the position on the biological surface compressed by the inflatable portion serving as the compression portion 106a. Therefore, even when the separated lower side portion of the pre-compression member 250 remains between the support portion 207a of the support body 207 and the biological surface, the separated lower side portion does not interfere with the inflation of the inflatable portion serving as the compression portion 106a.

According to the pre-compression member 250 in the present embodiment, as compared with the pre-compression member 150 (see FIG. 4) according to the second embodiment described above, in addition to the positions on both sides in the width direction B with respect to the compression portion 106a, the position in the backward direction C2 with respect to the compression portion 106a can be pre-compressed. Specifically, the pre-compression member 250 according to the present embodiment can pre-compress the biological surface by the lower frame portions 251c of the frame-shaped portions 260 located on both sides in the width direction B with respect to the compression portion 106a and the lower side interlocking piece 261a of the interlock portions 261 located in the backward direction C2 with respect to the compression portion 106a.

In other words, the pre-compression member 250 according to the present embodiment includes, as the protruding portions 51, a first protruding portion and a second protruding portion that are provided on both sides sandwiching the compression portion 106a of the compression device 201 in the plan view (see FIG. 12B). The first protruding portion according to the present embodiment is implemented by a lower end portion including the lower frame portion 251c of one of the two frame-shaped portions 260. In addition, the second protruding portion according to the present embodiment is implemented by a lower end portion including the lower frame portion 251c of the other one of the two frame-shaped portions 260. Further, the pre-compression member 250 according to the present embodiment further includes, as the protruding portion 51, a third protruding portion provided in a direction orthogonal to a direction (the width direction B in the present embodiment) in which the first protruding portion and the second protruding portion face each other with respect to the compression portion 106a of the compression device 201 in the plan view (see FIG. 12B). The third protruding portion according to the present embodiment is implemented by the lower side interlocking piece 261a of the interlock portion 261.

Accordingly, the pre-compression member 250 includes the first protruding portion, the second protruding portion, and the third protruding portion as the protruding portions 51, so that the pre-compression member 250 can perform pre-compression in a relatively wider range as compared with a case of using the pre-compression member 150 according to the second embodiment described above that includes only the first protruding portion and the second protruding portion. Therefore, pain felt by the patient can be reduced by the pre-compression performed by the protruding portions 51.

The pre-compression member 250 including the first protruding portion, the second protruding portion, and the third protruding portion is not limited to a shape according to the present embodiment. The pre-compression member 250 may have another shape including shapes of the first protruding portion, the second protruding portion, and the third protruding portion.

The pre-compression member 250 according to the present embodiment is attached to the compression device 201 in a manner of being freely movable within a predetermined range in the thickness direction A, and may be provided with the movement restriction mechanism as in the second embodiment described above.

Further, the pre-compression member 250 according to the present embodiment is separable in the thickness direction A, and may not be separable. That is, the pre-compression member 250 may not be detachable from the compression device 201. However, as in the present embodiment, the pre-compression member 250 is preferably detachable from the compression device 201. Accordingly, it is possible to solve a problem that the pre-compression member 250 becomes an obstacle after the compression device 201 is adhered to the biological surface.

Finally, an example of a living body compression method performed by the compression device 1 in the compression device set 100 according to the first embodiment, the compression device 101 in the compression device set 200 according to the second embodiment, and the compression device 201 in the compression device set 300 according to the third embodiment will be described. Here, for convenience of description, description is given using the compression device 101 according to the second embodiment, and the same applies to the compression device 1 according to the first embodiment and the compression device 201 according to the third embodiment.

By compressing the biological surface BS with the compression device 101, a perforation leading from the biological surface to a vein can be narrowed or obstructed without obstructing the vein. The perforation is formed by removing a sheath serving as the medical insertion member 1000 (see FIGS. 18A and 20) in a state of being inserted into a vein such as a femoral vein from the biological surface BS through a connective tissue. The compression device 101 can stop bleeding after removing the sheath serving as the medical insertion member 1000. First, the perforation formed after the medical insertion member 1000 is removed will be described with reference to FIGS. 18A and 18B. FIG. 18A shows a state in which the sheath serving as the medical insertion member 1000 is inserted into a femoral vein FV from the biological surface BS through a connective tissue CT. FIG. 18A shows three sheaths serving as the medical insertion member 1000, and the number of sheaths, for example, may be two or less, or may be four or more. FIG. 18B shows a state after the sheaths serving as the medical insertion members 1000 are removed from the state shown in FIG. 18A. As shown in FIG. 18B, the sheaths serving as the medical insertion members 1000 are removed, so that the perforations P are formed between the biological surface BS and the femoral vein FV. By using the compression device 101, the perforations P can be narrowed or obstructed without obstructing the femoral vein FV. Therefore, even when bleeding from a vein located at a deep position from the biological surface is stopped, bleeding can be stopped more efficiently without the need to narrow or obstruct the vein itself.

As described above, in a case of stopping bleeding from the vein, the bleeding can be stopped by narrowing or obstructing the perforations P (see FIG. 18B). On the other hand, for example, in a case of stopping bleeding from a femoral artery, even when only the perforations are obstructed, the blood leaks and spreads in the connective tissue CT (see FIGS. 18A and 18B), and thus the bleeding cannot be stopped. In the case of stopping bleeding from the femoral artery, it is necessary to take a large measure, such as a method of strongly compressing the artery itself until the artery is narrowed or obstructed, or a method of obstructing a hole in an artery wall.

Therefore, in a case of stopping the bleeding from the vein using the compression device 101, the biological surface BS is preferably compressed to a position at which a compression depth from the biological surface BS is 5 mm to 20 mm. By setting the compression depth within the above range, it is relatively easy to implement a compression state in which the perforations P (see FIG. 18B) are narrowed or obstructed without obstructing the vein. The compression depth can be, for example, preferably 5 mm to 15 mm, and more preferably 8 mm to 12 mm.

Further, in the case of stopping the bleeding from the vein using the compression device 101, the biological surface BS is preferably compressed at 10 g/cm² to 600 g/cm² from the biological surface BS. A compression pressure means a pressure after the sheath serving as the medical insertion member 1000 is removed. By setting the compression pressure within the above range, it is relatively easy to implement the compression state in which the perforations P (see FIG. 18B) are narrowed or obstructed without obstructing the vein. The compression pressure can be, for example, 50 g/cm² to 400 g/cm², and preferably 100 g/cm² to 300 g/cm².

The biological surface BS is preferably compressed along a direction orthogonal to an extending direction of the perforations P (see FIG. 18B). The phrase “compressed along a direction orthogonal to an extending direction of the perforations” means not only compressing only in the direction orthogonal to the extending direction of the perforations but also compressing in a direction inclined at an angle equal to or less than a predetermined angle (for example, 30 degrees or less) with respect to the direction orthogonal to the extending direction of the perforations. The compression device 101 can compress the biological surface BS along the direction orthogonal to the extending direction of the perforations P (see FIG. 18B).

Specifically, the inflatable portion serving as the compression portion 106a of the compression device 101 can inflate toward the direction inclined with respect to the thickness direction A, as described above. Accordingly, the biological surface can be compressed along the direction orthogonal to the extending direction of the perforations P (see FIG. 18B). Specifically, as shown in FIGS. 18A and 18B, the sheath serving as the medical insertion member 1000 is inserted not in a direction (a direction that is the same as the thickness direction A) orthogonal to the biological surface BS but in a direction inclined to one side with respect to the direction orthogonal to the biological surface BS. Therefore, as shown in FIG. 18B, the extending direction of the perforations P is also inclined with respect to the direction orthogonal to the biological surface BS. Therefore, when the compression portion 106a can be inflated in a direction (hereinafter, may be referred to as an “inclination direction F”) inclined to a side opposite to the extending direction of the perforations P with respect to the thickness direction A which is the direction orthogonal to the biological surface BS, the biological surface BS is relatively easily compressed along the direction orthogonal to the extending direction of the perforations P. Accordingly, it is easy to implement the compression device 101 that narrows or obstructs the perforations P without obstructing the vein such as the femoral vein FV in FIGS. 18A and 18B. FIG. 19 is a diagram showing the state in which the perforations P shown in FIG. 18B are narrowed or obstructed by the compression device 101. As shown in FIG. 19, the perforations P are easily narrowed or obstructed by the compression device 101 without further obstructing the vein such as the femoral vein FV.

Accordingly, in the case of stopping the bleeding from the vein using the compression device 101, the bleeding can be stopped by narrowing or obstructing the perforations P (see FIG. 18B) without obstructing the vein such as the femoral vein FV. In particular, by implementing the living body compression method using the compression device 101, it is possible to stop the bleeding by a method without compression performed by a hand of the health care worker or using a large-scale hemostasis device.

Compression to Biological Surface by Compression Device 101

As shown in FIG. 19, in the compression device 101, the inflatable portion serving as the compression portion 106a of the compression member 103 can compress the biological surface toward the inclination direction F inclined with respect to a vertical direction (in FIG. 19, the vertical direction is the same direction as the thickness direction A, and is an upper-lower direction in FIG. 19. Hereinafter, the direction is referred to as a “vertical direction”) perpendicular to the biological surface BS in a state in which the adhesion body 102 is adhered to the living body. Accordingly, as shown in FIG. 19, the perforations P can be rather easily narrowed or obstructed without obstructing the vein such as the femoral vein FV.

FIG. 20 is a front view of the state shown in FIG. 19 as viewed from the biological surface BS side. In other words, FIG. 20 is a front view of the biological surface BS at a position compressed by the compression device 101. Here, the phrase “front view of the biological surface at a position compressed by the compression device” means a state in which a portion of the biological surface to be compressed by the compression device is viewed from a direction perpendicular to the portion before being compressed. FIG. 20 is a front view of an inguinal region. In the front view shown in FIG. 20, a direction (see a white arrow “AR1” in FIG. 20) in which the biological surface BS is compressed faces an insertion direction G1 (see a white arrow “AR2” in FIG. 20) of the sheath from the biological surface BS toward the vein in an extending direction G of the perforations P. That is, the direction in which the compression device 101 compresses the biological surface BS faces the insertion direction G1 of the sheath in the front view shown in FIG. 20. Accordingly, the perforations P (see FIGS. 18B and 19) can be rather easily narrowed or obstructed without obstructing the vein such as the femoral vein FV.

In other words, as shown in FIG. 19, the extending direction G of the perforations P is inclined with respect to the biological surface BS and is also inclined with respect to the vertical direction (upper-lower direction in FIG. 19) perpendicular to the biological surface BS. In addition, as shown in FIG. 19, a direction in which the biological surface BS is compressed by the compression device 101 is also inclined with respect to the biological surface BS and also inclined with respect to the vertical direction (upper-lower direction in FIG. 19) perpendicular to the biological surface BS. Further, as shown in FIG. 19, the extending direction G of the perforations P is inclined to a side opposite to the inclination direction F serving as the direction in which the biological surface is compressed by the compression device 101 with respect to the vertical direction (upper-lower direction in FIG. 19). That is, the compression device 101 compresses the biological surface such that the compression direction intersects with the extending direction G of the perforations P. Accordingly, the perforations P can be efficiently narrowed or obstructed.

The compression device set and the adhering method according to the present disclosure are not limited to the specific configurations and steps described in the above embodiments, and various modifications, changes, and combinations may be made without departing from the scope of the claims. It is expressly intended that all such changes, modifications and equivalents which fall within the scope of the claims are embraced by the claims.

What is claimed is:

1. A compression device set comprising:
a compression device configured to be adhered to a biological surface and to compress the biological surface in a state of being adhered to the biological surface;
a pre-compression member configured to be attached to the compression device and configured to compress, before the compression device is adhered, the biological surface to which the compression device is to be adhered;
the compression device including an adhesion surface configured to be adhered to the biological surface and a compression portion configured to compress the biological surface in a compression region, the compression region being a position sandwiched by the adhesion surface or a position surrounded by the adhesion surface in a plan view viewed along a direction orthogonal to the adhesion surface; and
wherein the pre-compression member includes a protruding portion configured to protrude in a direction of the biological surface from the adhesion surface of the compression device and configured to compress the biological surface before the adhesion surface is adhered to the biological surface in the compression region.

2. The compression device set according to claim 1, wherein the pre-compression member is configured to attach to the compression device in a manner of being relatively movable in the direction orthogonal to the adhesion surface.

3. The compression device set according to claim 2, wherein the compression device and the pre-compression member include a movement restriction mechanism configured to limit movement of the pre-compression member relative to the compression device in the direction orthogonal to the adhesion surface and configured to fix a minimum protrusion amount of the protruding portion.

4. The compression device set according to claim 3, wherein the movement restriction mechanism is configured to adjust the minimum protrusion amount of the protruding portion in a plurality of stages.

5. The compression device set according to claim 1, wherein the pre-compression member is configured to be attachable to and detachable from the compression device.

6. The compression device set according to claim 1, wherein the pre-compression member includes, as the protruding portion, a first protruding portion and a second protruding portion that are provided on both sides sandwiching the compression portion of the compression device in the plan view viewed along the direction orthogonal to the adhesion surface.

7. The compression device set according to claim 6, wherein the pre-compression member further includes, as the protruding portion, a third protruding portion provided on the compression portion of the compression device in a direction orthogonal to a direction in which the first protruding portion and the second protruding portion face each other in the plan view viewed along the direction orthogonal to the adhesion surface.

8. The compression device set according to claim 1, wherein
the compression device further includes an adhesion body having the adhesion surface, and a compression member fixed to a surface opposite to the adhesion surface of the adhesion body;
the compression member including an inflatable portion constituting the compression portion and configured to be inflatable, and a support portion configured to support the inflatable portion, the support portion defining a through hole penetrating in the direction orthogonal to the adhesion surface; and
wherein the protruding portion of the pre-compression member protrudes from the adhesion surface through the through hole.

9. A compression device set comprising:
a compression device configured to be adhered to a biological surface;
a pre-compression member to compress, before the compression device is adhered, the biological surface to which the compression device is to be adhered;
the compression device including an adhesion surface configured to be adhered to the biological surface and a compression portion configured to compress the biological surface in a compression region; and
wherein the pre-compression member includes a protruding portion configured to protrude in a direction of the biological surface from the adhesion surface of the compression device and configured to compress the biological surface before the adhesion surface is adhered to the biological surface in the compression region.

10. The compression device set according to claim 9, wherein the compression region is a position sandwiched by the adhesion surface in a plan view viewed along a direction orthogonal to the adhesion surface.

11. The compression device set according to claim 9, wherein the compression region is a position surrounded by the adhesion surface in a plan view viewed along a direction orthogonal to the adhesion surface.

12. The compression device set according to claim 9, wherein the pre-compression member is configured to attach to the compression device in a manner of being relatively movable in the direction orthogonal to the adhesion surface.

13. The compression device set according to claim 12, wherein the compression device and the pre-compression member include a movement restriction mechanism configured to limit movement of the pre-compression member relative to the compression device in the direction orthogonal to the adhesion surface and configured to fix a minimum protrusion amount of the protruding portion.

14. The compression device set according to claim 13, wherein the movement restriction mechanism is configured to adjust the minimum protrusion amount of the protruding portion in a plurality of stages.

15. The compression device set according to claim 9, wherein the pre-compression member is configured to be attachable to and detachable from the compression device.

16. The compression device set according to claim 9, wherein the pre-compression member includes, as the protruding portion, a first protruding portion and a second protruding portion that are provided on both sides sandwiching the compression portion of the compression device in the plan view viewed along the direction orthogonal to the adhesion surface.

17. The compression device set according to claim 16, wherein the pre-compression member further includes, as the protruding portion, a third protruding portion provided on the compression portion of the compression device in a direction orthogonal to a direction in which the first protruding portion and the second protruding portion face each other in the plan view viewed along the direction orthogonal to the adhesion surface.

18. The compression device set according to claim 9, wherein
the compression device further includes an adhesion body having the adhesion surface, and a compression member fixed to a surface opposite to the adhesion surface of the adhesion body;
the compression member including an inflatable portion constituting the compression portion and configured to be inflatable, and a support portion configured to support the inflatable portion, the support portion defining a through hole penetrating in the direction orthogonal to the adhesion surface; and
wherein the protruding portion of the pre-compression member protrudes from the adhesion surface through the through hole.

19. An adhering method for adhering a compression device configured to compress a biological surface in a state of being adhered to the biological surface to the biological surface in a state of pre-compressing the biological surface, the compression device including an adhesion surface configured to be adhered to the biological surface, and a compression portion configured to compress the biological surface in a compression region, the compression region being a position sandwiched by the adhesion surface or a position surrounded by the adhesion surface in a plan view viewed along a direction orthogonal to the adhesion surface, the adhering method comprising:
adhering, in the compression region, the adhesion surface of the compression device to the biological surface in a state of compressing the biological surface by a protruding portion of a pre-compression member attached to the compression device, the protruding portion protruding from the adhesion surface.

20. The method according to claim 19, further comprising:
attaching the pre-compression member to the compression device in a manner of being relatively movable in the direction orthogonal to the adhesion surface.
